(19) United States
(12) Patent Application Publication
(10) Pub. No.: US 2017/0038381 A1
(43) Pub. Date: Feb. 9, 2017

(54) METHODS AND COMPOSITIONS FOR IDENTIFYING PATIENT POPULATIONS FOR DIAGNOSIS AND TREATMENT OF TLR4-DEPENDENT DISORDERS

(71) Applicant: NovImmune SA, Geneva (CH)

(72) Inventors: Emmanuel Monnet, Geneva (CH); Limin Shang, Geneva (CH)

(21) Appl. No.: 15/229,816

(22) Filed: Aug. 5, 2016

Related U.S. Application Data
(60) Provisional application No. 62/201,918, filed on Aug. 6, 2015.

(51) Int. Cl. G01N 33/564; G01N 33/68

(52) U.S. Cl. CPC G01N 33/564; G01N 33/6854; G01N 2800/102

(57) ABSTRACT

This invention relates generally to methods and compositions for identifying patient populations for diagnosis and treatment of Toll-like Receptor 4 (TLR4)-dependent disorders. In particular, the invention relates to detecting levels of anti-citrullinated protein antibodies (ACPA) and citrullinated peptides to identify patients having a TLR4-dependent disease and to identify patients who are likely to respond to anti-TLR4 therapy. The invention also relates to methods of treating, delaying the progression of, or otherwise ameliorating a symptom of a disorder in patients with elevated levels of ACPA and citrullinated peptides using agents that interfere with or otherwise antagonize TLR-4 signaling, including neutralizing anti-TLR4 antibodies.

### RELATED APPLICATIONS

This application claims the benefit of U.S. Provisional Application No. 62/201,918, filed Aug. 6, 2015, the contents of which are incorporated herein by reference in their entirety.

### INCORPORATION OF SEQUENCE LISTING

The contents of the text file named “NOVI041001US_ST25.txt”, which was created on Aug. 5, 2016 and is 115 KB in size, are hereby incorporated by reference in their entirety.

### FIELD OF THE INVENTION

This invention relates generally to methods and compositions for identifying patient populations for diagnosis and treatment of Toll-like Receptor 4 (TLR4)-dependent disorders. In particular, the invention relates to detecting levels of anti-citrullinated protein antibodies (ACPA) and/or antibodies against specific citrullinated proteins and/or peptides to identify patients having a TLR4-dependent disease and to identify patients who are likely to respond to anti-TLR4 therapy. The invention also relates to methods of treating, delaying the progression of, or otherwise ameliorating a symptom of a disorder in patients with elevated levels of ACPA and/or antibodies against specific citrullinated proteins and/or peptides using agents that interfere with or otherwise antagonize TLR-4 signaling, including neutralizing anti-TLR4 antibodies.

### BACKGROUND OF THE INVENTION

Toll receptors, first discovered in Drosophila, are type I transmembrane protein having leucine-rich repeats (LRRs) in the extracellular portion of the protein, and one or two cysteine-rich domains. The mammalian homologs of the Drosophila Toll receptors are known as “Toll-like receptors” (TLRs). TLRs play a role in innate immunity by recognizing microbial particles and activating immune cells against the source of these microbial particles. In humans, eleven Toll-like receptors, TLRs 1-11, have been identified and are characterized by the homology of their intracellular domains to that of the IL-1 receptor, and by the presence of extracellular leucine-rich repeats. The different types of TLRs are activated by different types of microbial particles. For example, TLR4 is primarily activated by lipopolysaccharide (LPS). TLR4 has been shown to associate with an accessory protein, myeloid differentiation protein-2 (MD-2). This protein has been found to interact directly with TLR4, and MD-2 has the ability to enable post-translational modifications of TLR4, as well as facilitate its transport to the cell surface. TLR4 and MD-2 form a complex on the cell surface.

TLR4 has been implicated in a number of disorders; and anti-TLR4 agents are being developed as therapeutic agents. Not all patients respond to current standard of care therapies. Accordingly, there exists a need for compositions and methods for use in identifying patients that are likely candidates for a particular treatment, for example, treatment with a particular anti-TLR4 therapy.

### SUMMARY OF THE INVENTION

The compositions and methods provided herein are useful in identifying or otherwise refining a patient population suffering from a disorder, where the patient has an elevated level of one or more TLR4 ligands or other TLR4-related biomarkers. These patients are identified as suitable candidates for treatment with an agent (e.g., antibodies or other polypeptide-based therapeutics, peptide-based therapeutics, small molecule inhibitors, nucleic acid-based therapeutics and derivatives thereof) that interferes with or otherwise antagonizes TLR4 signaling and neutralizes at least one biological activity of TLR4, alone or in the context of the accessory protein MD-2 as the TLR4/MD-2 complex.

In some patients suffering from or suspected of suffering from a disorder, fluids and other biological samples contain elevated levels of TLR4 ligands such as immune complexes containing ACPA and citrullinated proteins and/or peptides. These TLR4 ligands stimulate cells to produce pro-inflammatory cytokines. However, use of an anti-TLR4 antagonist that interferes with or otherwise antagonizes TLR4 signaling, e.g., a neutralizing anti-TLR4 antibody or other anti-TLR4 agent, is shown herein to block this stimulation in patients exhibiting an elevated level of expression for one or more TLR4 ligands and/or other related biomarkers. Thus, the compositions and methods are useful in treating, delaying the progression of or otherwise ameliorating a symptom of a disorder that is dependent on, driven by, or otherwise associated with TLR4 signaling, aberrant, e.g., elevated, TLR4 ligand expression and/or activity, aberrant pro-inflammatory cytokine production, and/or combinations thereof, by administering an anti-TLR4 antagonist, e.g., a neutralizing anti-TLR4 antibody or other polypeptide-based therapeutic, a peptide-based therapeutic, a small molecule inhibitor, a nucleic acid-based therapeutic and derivatives thereof, to patients exhibiting an elevated level of expression for one or more TLR4 ligands and/or related biomarkers. Patients that are likely suitable candidates for treatment with the anti-TLR4 antagonist, e.g., neutralizing anti-TLR4 antibody such as those described herein, are identified by detecting the level of one or more TLR4 ligands or other related biomarkers.

Suitable TLR4 ligands and other related biomarkers for use in identifying likely candidates include ACPA and/or antibody against one or more specific citrullinated proteins and/or peptides. In some embodiments, the citrullinated peptide is derived from citrullinated fibrinogen (cFb). In some embodiments, the citrullinated peptide is derived from citrullinated fibrinogen alpha (cFbα). In some embodiments, the citrullinated peptide is derived from citrullinated fibrinogen beta (cFbβ). In some embodiments, the citrullinated peptide is derived from citrullinated histone. In some embodiments, the citrullinated peptide is derived from citrullinated histone 2A.

In some embodiments, the citrullinated peptide comprises the amino acid sequence NTKESSSHHPGIAEFPS-Cit-GK (SEQ ID NO: 1), where Cit=citrulline. This peptide is referred to herein as cFbα 556-575.

In some embodiments, the citrullinated peptide comprises the amino acid sequence HHPGIAEFPS-Cit-GKSSSYSKQF (SEQ ID NO: 2), where Cit=citrulline. This peptide is referred to herein as citFbβ 563-583.

In some embodiments, the citrullinated peptide comprises the amino acid sequence MSG-Cit-GKQGGKA-Cit-AKAKS-Cit-SS (SEQ ID NO: 3), where Cit=citrulline. This peptide is referred to herein as citH2A 1-20.

In the methods provided herein, the level of expression of ACPA and/or antibody against one or more specific citrullinated protein and/or peptides is detected in a biological sample. In some embodiments, the level of expression of ACPA and/or antibody against one or more specific citrullinated protein and/or peptides is detected in a combination of biological samples. In some embodiments, the biological sample is synovial fluid. In some embodiments, the biological sample is blood or is derived from blood. In some embodiments, the biological sample is serum. In some embodiments, the biological sample is a combination of synovial fluid and serum samples.

Patients with elevated levels of one or more of these markers are identified as suitable candidates for therapy with one or more anti-TLR4 antagonists, e.g., a neutralizing anti-TLR4 antibody described herein. As used herein, the phrase “elevated level of expression” refers to a level of expression that is greater than a baseline level of expression of ACPA and/or antibody against one or more specific citrullinated protein and/or peptides in a sample from a patient that is not suffering from or suspected of suffering from a disorder or other control sample. In some embodiments, the elevated level of expression of ACPA and/or antibody against one or more specific citrullinated protein and/or peptides is a significant level of elevation.

Patients where treatment with an anti-TLR4 antibody was able to block, partially or totally, cytokine production in rheumatoid arthritis monocytes are identified as “responders,” while patients where treatment with an anti-TLR4 antibody did not block, partially or totally, cytokine production are identified as “non-responders.”

In addition to detecting the level of ACPA and/or antibody against one or more specific citrullinated protein and/or peptides, suitable patients for treatment with an anti-TLR4 antagonist can also be identified by evaluating any of a number of additional biological and clinical parameters that will improve the sensitivity and specificity of the biomarker for identifying or otherwise refining the patient population. Alternatively, these additional biological and clinical parameters can be used alone as a means for identifying patients that are suitable candidates for treatment with an anti-TLR4 antagonist or other suitable therapy. These biological and clinical parameters include, by way of non-limiting example, any of the following: rheumatoid factor levels, C-reactive protein (CRP) levels, blood cells count, presence of TLR4 receptor on blood cell subpopulations, TLR4 polymorphisms, human leukocyte antigen (HLA) polymorphisms, peptidyl arginine deiminase (PAD) enzymes and PAD enzyme polymorphisms, Fcγ Receptor IIa (FcγIIa) polymorphisms, MD-2 levels, soluble CD14 levels, baseline patient demographic data (e.g., body mass index (BMI), sex, age, etc.) and/or patient medical history (e.g., disability assessment schedule (DAS 28) at diagnosis, DAS 28 at treatment initiation, duration of disease, age at disease onset, response to prior treatments based on DAS28, American College of Rheumatology (ACR) and/or European League Against Rheumatism (EULAR) response criteria, etc.).

Disorders that are useful with the compositions and methods of the invention include any disorder where aberrant, e.g., elevated, TLR4 expression and/or activity, with aberrant TLR4/MD-2 activation and/or aberrant TLR4 ligand activity (e.g., aberrant stimulation of pro-inflammatory cytokine production such as aberrant stimulation of IL-6, TNFα and/or IL-8 production). For example, some TLR4 ligands are believed to be associated with various disorders. By way of non-limiting example, LPS is known to be associated with disorders such as sepsis, acute lung injury, and/or RA; Tenascin C is known to be associated with disorders such as arthritis, hepatic and/or cardiac ischemial reperfusion; HMGB1 is known to be associated with disorders such as RA, Osteoarthritis (OA), ischemia/reperfusion, Type 1 diabetes, islet transplantation, lupus and/or sepsis; S100A8/A9 is known to be associated with disorders such as RA, OA, juvenile idiopathic arthritis (JIA), diabetes, transplant rejection, lupus, atherosclerosis, sepsis and/or cancer; citrullinated fibrinogen is known to be associated with disorders such as RA and atherosclerosis; ACPA is known to be associated with disorders such as RA, psoriatic arthritis, systemic lupus erythematosus (SLE), Sjogren's syndrome, Alzheimer disease and/or atherosclerosis.

By way of non-limiting examples, the methods and compositions provided herein are suitable for diagnosing and/or treating disorders such as autoimmune and/or inflammatory disorders. Suitable autoimmune and/or inflammatory disorders include, by way of non-limiting example, autoimmune and/or inflammatory disorders associated with aberrant TLR4 signaling, autoimmune and/or inflammatory disorders associated with aberrant, e.g., elevated, TLR4 ligand expression and/or activity, autoimmune and/or inflammatory disorders associated with aberrant pro-inflammatory cytokine production, and combinations thereof.

In some embodiments, the disorder is an arthritis condition, including by way of non-limiting example, RA, Osteoarthritis (OA), psoriatic arthritis or juvenile idiopathic arthritis (JIA). In some embodiments, the disorder is rheumatoid arthritis (RA). In some embodiments, the disorder is cancer. In some embodiments, the disorder is inflammatory bowel disease (IBD). In some embodiments, the disorder is atherosclerosis. In some embodiments, the disorder is associated with ischemial reperfusion, including by way of non-limiting example, hepatic and/or cardiac ischemia/reperfusion. In some embodiments, the disorder is sepsis. In some embodiments, the disorder is acute lung injury. In some embodiments, the disorder is Type 1 diabetes. In some embodiments, the disorder is associated with islet transplantation. In some embodiments, the disorder is lupus. In some embodiments, the disorder is associated with transplant rejection or other disorder associated with cell, tissue and/or organ transplant. In some embodiments, the disorder is systemic lupus erythematosus (SLE). In some embodiments, the disorder is Sjogren's syndrome. In some embodiments, the disorder is Alzheimer's disease.

Once patients are identified as having an elevated level of ACPA and/or antibody against one or more specific citrullinated protein and/or peptides, they are then treated with an anti TLR4 antagonist. For example, the anti TLR4 antagonist is a neutralizing anti TLR4 antibody or an immunologically active (e.g., antigen binding) fragment thereof. Suitable neutralizing antiTLR4 antibodies include any of the anti-TLR4 antibodies described herein and other antibodies with increased affinity for Fc receptor (FcR) and/or increased avidity for cell surface binding through interaction with FcR.

In some embodiments, the antibody or immunologically active fragment thereof that binds TLR4 comprises a variable heavy chain complementarity determining region 1 (VH CDR1) comprising an amino acid sequence at least 90%, 92%, 95%, 96%, 97% 98%, 99% or more identical to the amino acid sequence of GGYSWH (SEQ ID NO: 139); a VH CDR2 region comprising an amino acid sequence at least 90%, 92%, 95%, 96%, 97% 98%, 99% or more identical to the amino acid sequence of YIHYSGYTDFNPSLKT (SEQ ID NO: 140); and a VH CDR3 region comprising an amino acid sequence at least 90%, 92%, 95%, 96%, 97% 98%, 99% or more identical to the amino acid sequence of KDPSDAFPY (SEQ ID NO: 141); a variable light chain complementarity determining region 1 (VL CDR1) region comprising an amino acid sequence at least 90%, 92%, 95%, 96%, 97% 98%, 99% or more identical to the amino acid sequence of RASQSISDHLH (SEQ ID NO: 4); a VL CDR2 region comprising an amino acid sequence at least 90%, 92%, 95%, 96%, 97% 98%, 99% or more identical to the amino acid sequence of YASHAIS (SEQ ID NO: 5); and a VL CDR3 region comprising an amino acid sequence at least 90%, 92%, 95%, 96%, 97% 98%, 99% or more identical to the amino acid sequence of QQGHSFPLT (SEQ ID NO: 6). In some embodiments, the antibody or immunologically active fragment thereof that binds TLR4 further comprises an amino acid sequence at least 90%, 92%, 95%, 96%, 97% 98%, 99% or more identical to the heavy chain variable amino acid sequence QVQLQESGPGLVKPSDTLSLTCAVSGYSITGGYSWHWIRQPPGKGLEWMGYIHYSGYT DFNPSLKTRITISRDTSKNQFSLKLSSVTAVDTAVYYCARKDPSDAFPYWGQGTLVTVSS (SEQ ID NO: 7) and an amino acid sequence at least 90%, 92%, 95%, 96%, 97% 98%, 99% or more identical to the light chain variable amino acid sequence EIVLTQSPDFQSVTPKEKVTITCRASQSISDHLHWYQQKPDQSPKLLIKYASHAISGVPSR FSGSGSGTDFTLTINSLEAEDAATYYCQQGHSFPLTFGGGTKVEIK (SEQ ID NO: 8). In some embodiments, the antibody or immunologically active fragment thereof that binds TLR4 further comprises an amino acid sequence at least 90%, 92%, 95%, 96%, 97%, 98%, 99% or more identical to the heavy chain amino acid sequence MGWSWIFLFLLSGTAGVHCQVQLQESGPGLVKPSDTLSLTCAVSGYSITGGYSWHWIR QPPGKGLEWMGYIHYSGYTDFNPSLKTRITISRDTSKNQFSLKLSSVTAVDTAVYYCAR KDPSDAFPYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVDKRVE PKSCDKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSSKAFPAPIE KTISKAKGQPREPQVYTLPPSREEMTKNQVSLTCLVKGFYPSDIAVEWESNGQPENNYK TTPPVLDSDGSFFLYSKLTVDKSRWQQGNVFSCSVMHEALHNHYTQKSLSLSPGK (SEQ ID NO: 9) and an amino acid sequence at least 90%, 92%, 95%, 96%, 97% 98%, 99% or more identical to the light chain amino acid sequence MEWSWVFLFFLSVTTGVHSEIVLTQSPDFQSVTPKEKVTITCRASQSISDHLHWYQQKPD QSPKLLIKYASHAISGVPSRFSGSGSGTDFTLTINSLEAEDAATYYCQQGHSFPLTFGGGT KVEIKRTVAAPSVFIFPPSDEQLKSGTASVVCLLNNFYPREAKVQWKVDNALQSGNSQE SVTEQDSKDSTYSLSSTLTLSKADYEKHKVYACEVTHQGLSSPVTKSFNRGEC (SEQ ID NO: 10).

In some embodiments, anti-TLR4 antibody or immunologically active fragment thereof is or is derived from an antibody as described in PCT/IB2005/004206, filed Jun. 14, 2005 and published as WO 20071110678, the contents of which are hereby incorporated by reference in their entirety.

In some embodiments, anti-TLR4 antibody or immunologically active fragment thereof is or is derived from an antibody as described in PCT application PCT/IB2008/003978, filed May 14, 2008 and published as WO 2009/101479, the contents of which are hereby incorporated by reference in their entirety.

In some embodiments, anti-TLR4 antibody or immunologically active fragment thereof is or is derived from the anti-TLR4 antibody known as HTA125, which is described, for example, in Shimazu, et al., J. Exp. Med., val. 189:1777-1782 (1999); Nijhuis et al., Clin Diag. Lab. Immunol., val. 10(4): 558-63 (2003); and Pivarcsi et al., Intl. Immunopharm., vol. 15(6):721-730 (2003), the contents of each of which are hereby incorporated by reference in their entirety.

In some embodiments, the anti-TLR4 antibody or immunologically active fragment thereof is or is derived from a domain antibody such as, for example, the domain antibodies that bind TLR4 described in PCT application PCT/EP2009/055926, filed May 15, 2009 and published as WO 2009/13848, the contents of which are hereby incorporated by reference in their entirety.

In some embodiments, the anti-TLR4 antibody or immunologically active fragment thereof is or is derived from monoclonal antibodies recognizing human and/or cynomolgus monkey TLR4/MD-2 receptor expressed on the cell surface. The antibodies are capable of blocking, e.g., neutralizing, receptor activation and subsequent intracellular signaling induced TLR4 ligands, e.g., LPS or any other TLR4 ligand described herein. Antibodies of the invention include antibodies that bind human and cynomolgus monkey TLR4/MD-2 receptor complex and also bind TLR4 independently of the presence of MD-2.

In some embodiments, the anti-TLR4 antibody or immunologically active fragment thereof interferes with or otherwise antagonizes signaling via human and/or cynomolgus monkey TLR4/MD-2 receptor expressed on the cell surface, e.g., by blocking receptor activation and subsequent intracellular signaling induced by LPS. Exemplary monoclonal antibodies of these embodiments include: 1A1, 1A6, 1B12, 1C7, 1C10, 1C12, 1D10, 1E11, 1E11 N103D, 1G12, 1E11.C1, 1E11.C2, 1E11.C3, 1E11.C4, 1E11.C5, 1E11.C6, 1E11.E1, 1E11.E2, 1E11.E3, 1E11.E4, 1E11.E5, 1E11.C2E1, 1E11.C2E3, 1E11.C2E4 and 1E11.C2E5.

These antibodies have distinct specificities. Some antibodies show specificity for both the human and cynomolgus monkey TLR4 and/or both the human and cynomolgus monkey TLR4/MD-2 receptor complex, and they have been shown to inhibit receptor activation and subsequent intracellular signaling via LPS. For example, 1C12, 1E11, 1E11 N103D, 1E11.C1, 1E11.C2, 1E11.C3, 1E11.C4, 1E11.C5, 1E11.C6, 1E11.C2E1, 1E11.C2E2, 1E11.C2E3, 1E11.C2E4 and 1E11.C2E5 bind both human and cynomolgus monkey TLR4 independently of the presence of human or cynomolgus monkey MD-2. 1A1, 1A6, 1B12, 1C7, 1C10, 1D10 and 1G12 only bind to cynomolgus monkey TLR4 independently of the presence of cynomolgus monkey MD-2. 1E11.E1, 1E11.E2, 1E11.E3, 1E11.E4 and 1E11.E5 bind only to human TLR4 independently of the presence of human MD-2.

The humanized antibodies of the invention contain a heavy chain variable region having an amino acid sequence shown herein. The humanized antibodies of the invention contain a light chain variable region having an amino acid sequence shown herein.

The three heavy chain CDRs include an amino acid sequence at least 90%, 92%, 95%, 97% 98%, 99% or more identical to a variable heavy chain complementarity determining region 1 (VH CDR1, also referred to herein as CDRH1) amino acid sequence selected from the group consisting of G(F/Y)PI(R/G/W)(Y/F/G)GYS (SEQ ID NO: 14), GYSITGGYS (SEQ ID NO: 15); GFPIRYGYS (SEQ ID NO: 16); GYPIRFGYS (SEQ ID NO: 17); GYPIRHGYS (SEQ ID NO: 18); GFPIGQGYS (SEQ ID NO: 19); GYPIWGGYS (SEQ ID NO: 20) and GYPIGGGYS (SEQ ID NO: 21), a variable heavy chain complementarity determining region 2 (VH CDR2, also referred to herein as CDRH2) amino acid sequence of IHYSGYT (SEQ ID NO: 22); and a variable heavy chain complementarity determining region 3 (VH CDR3, also referred to herein as CDRH3) amino acid sequence selected from the group consisting of ARKDSG(N/Q/D/E)X₁X₂PY. (SEQ ID NO: 23) where X₁ and X₂ are each independently any hydrophobic amino acid, ARKDSGNYFPY (SEQ ID NO: 24); ARKDSGRLLPY (SEQ ID NO: 25); ARKDSGKWLPY (SEQ ID NO: 26); ARKDSGHLMPY (SEQ ID NO: 27); ARKDSGHNYPY (SEQ ID NO: 28); ARKDSGKNFPY (SEQ ID NO: 29); ARKDSGQLFPY (SEQ ID NO: 30); ARKDSGHNLPY (SEQ ID NO: 31); ARKDSGDYFPY (SEQ ID NO: 32) and ARKDSGRYWPY (SEQ ID NO: 33). The three light chain CDRs include an amino acid sequence at least 90%, 92%, 95%, 97% 98%, 99% or more identical to a variable light chain complementarity determining region 1 (VL CDR1, also referred to herein as CDRL1) amino acid sequence of QSISDH (SEQ ID NO: 34); a variable light chain complementarity determining region 2 (VL CDR2, also referred to herein as CDRL2) amino acid sequence of YAS (SEQ ID NO: 35); and a variable light chain complementarity determining region 3 (VL CDR3, also referred to herein as CDRL3) amino acid sequence selected from the group consisting of QQG(Y/N)(D/E)(F/Y)PXT (SEQ ID NO: 36) where X is any hydrophobic amino acid, QQGHSFPLT (SEQ ID NO: 6); QQGNDFPVT (SEQ ID NO: 37); QQGYDEPFT (SEQ ID NO: 38); QQGYDFPFT (SEQ ID NO: 39); QQGYDYPFT (SEQ ID NO: 40) and QQGYEFPFT (SEQ ID NO: 41). The antibodies bind to human and cynomolgus monkey TLR4/MD-2 complex, to human and cynomolgus TLR4 when not complexed with human and cynomolgus MD-2, to human TLR4/MD-2 complex, to human TLR4 when not complexed with human MD-2, to cynomolgus monkey TLR4/MD-2 complex or cynomolgus TLR4 when not complexed with cynomolgus MD-2.

The anti-TLR4 antibodies of the invention also include antibodies that include a heavy chain variable amino acid sequence that is at least 90%, 92%, 95%, 97%, 98%, 99% or more identical an amino acid sequence shown herein, and/or a light chain variable amino acid that is at least 90%, 92%, 95%, 97%, 98%, 99% or more identical an amino acid sequence shown herein.

In some embodiments, the anti-TLR4 antibodies described herein also include at least one specific amino acid substitution within, for example, an Fc region or an FcR binding fragment thereof (e.g., a polypeptide having amino acid substitutions within an IgG constant domain) such that the modified antibody elicits alterations in antigen-dependent effector function while retaining binding to antigen as compared to an unaltered antibody. For example, the altered antibodies elicit the prevention of proinflammatory mediator release. In a preferred embodiment, the altered antibodies are human and of the IgG1 isotype.

The anti-TLR4 antibodies of the invention include an altered antibody in which at least one amino acid residue in the constant region of the Fc portion of the antibody has been modified. For example, at least one amino acid in the CH2 domain of the Fc portion has been replaced by a different residue, i.e., an amino acid substitution. In the altered antibodies described herein, one or more of the amino acid residues that correspond to residues 325, 326 and 328 is substituted with a different residue as compared to an unaltered antibody. The numbering of the residues in the gamma heavy chain is that of the EU index (see Edelman, G. M. et al., 1969; Kabat, E, A., T. T. Wu, H. M. Perry, K. S. Gottesman, and C. Foeller., 1991. Sequences of Proteins of Immunological Interest, 5th Ed. U.S. Dept. of Health and Human Services, Bethesda, Md., NIH Publication n. 91-3242). In a preferred embodiment, EU amino acid position 325 of the gamma heavy chain constant region is substituted with serine, and EU amino acid position 328 of the gamma heavy chain constant region is substituted with phenylalanine, such that the EU positions 325 to 328 of the gamma heavy chain constant region of the altered human IgG1 antibody comprise the amino acid sequence SKAF (SEQ ID NO: 13).

The present invention also provides methods of treating or preventing pathologies associated with aberrant TLR4/MD-2 activation, aberrant TLR4 signaling, aberrant, e.g., elevated, TLR4 ligand expression and/or activity, aberrant pro-inflammatory cytokine production, and combinations thereof, or alleviating a symptom associated with such pathologies, by identifying a patient suitable for therapy with a neutralizing anti-TLR4 agent, e.g., a neutralizing anti-TLR4 antibody, and administering the agent, e.g., a monoclonal antibody of the invention (e.g., a murine monoclonal or humanized monoclonal antibody) to a subject in which such treatment or prevention is desired. The subject to be treated is, e.g., human. The monoclonal antibody is administered in an amount sufficient to treat, prevent or alleviate a symptom associated with the pathology. The amount of monoclonal antibody sufficient to treat or prevent the pathology in the subject is, for example, an amount that is sufficient to reduce TLR4 ligand-induced production of one or more pro-inflammatory cytokines (e.g., IL-6, IL-8, TNFα). As used herein, the term “reduced” refers to a decreased production of a pro-inflammatory cytokine in the presence of a monoclonal antibody of the invention, wherein the production is, for example, local pro-inflammatory cytokine production (e.g., at a site of inflamed tissue) or systemic pro-inflammatory cytokine production. TLR4 ligand-induced production of a pro-inflammatory cytokine is decreased when the level of pro-inflammatory cytokine production in the presence of a monoclonal antibody of the invention is greater than or equal to 5%, 10%, 20%, 25%, 30%, 40%, 50%, 60%, 70%, 75%, 80%, 90%, 95%, 99%, or 100% lower than a control level of pro-inflammatory cytokine production (i.e., the level of pro-inflammatory cytokine production in the absence of the monoclonal antibody). Level of pro-inflammatory cytokine production is measured. Those skilled in the art will appreciate that the level of pro-inflammatory cytokine production can be measured using a variety of assays, including, for example, the methods described herein as well as commercially available ELISA kits.

Pharmaceutical compositions according to the invention can include an anti-TLR4 antibody of the invention and a carrier. These pharmaceutical compositions can be included in kits, such as, for example, diagnostic kits.

The invention also provides kits for practicing any of the methods provided herein. For example, in some embodiments, the kits include a detection reagent specific for ACPA and/or antibody against one or more specific citrullinated protein and/or peptides and a means for detecting the detection reagent.

### BRIEF DESCRIPTION OF THE DRAWINGS

FIGS. 1A, 1B, 1C, and 1D are a series of graphs depicting that treatment with the anti-TLR4 antibody referred to herein as NI-0101 blocks IL-6 (A), TNFα (B), IL-10 (C) and IL-8 (D) production from pooled rheumatoid arthritis synovial fluid (RASF)-stimulated monocytes isolated from rheumatoid arthritis (RA) patients. TLR4 signaling was blocked with anti-human TLR4 monoclonal antibody, NI-0101. Representative data shown for monocytes obtained from 1 of 7 RA patient donors. Data are presented as mean+/−SEM. The Mann-Whitney's U test was performed to analyze difference among groups. ** p<0.01, *** p<0.001.

FIGS. 2A and 2B are a series of graphs depicting the heterogeneous capacity of RASF samples to stimulate cytokine production and respond to TLR4 blockade. RASF samples from patients (Pat) were classified as NI-0101 responders (R) if NI-0101 was able to block (partially or totally) RASF-induced IL6 production from RA monocytes. Others were classified as NI-0101 non-responders (NR). Representative examples of non-responders RASF (Pat#13, #35) and responders RASF (Pat#27, #18) are depicted. Of the 36 RASF samples tested, 18 were classified as NI-0101 responders (50%) and 18 as NI-0101 responders (50%). Mann-Whitney's U test was performed to analyze difference among groups. *** p<0.001, * p<0.05.

FIGS. 3A, 3B, 3C, 3D, 3E, and 3F are a series of graphs depicting the expression levels of ACPA and TLR4 ligands in the synovial fluid samples of non RA and RA patients and their correlation with NI-0101 response. A-C, Expression levels of ACPA (A), HMGB1 (B) and S100A8/A9 (C) in synovial fluids from non-RA subjects (non-RASF; n=4 samples) and RA patients (RASF; n=36 samples). D-F, Correlation of levels of ACPA (D) and TLR4 ligands (E & F) with NI-0101 response. RASF samples were classified as NI-0101 non-responders (NR) or NI-0101 responders (R) according to the definition in FIG. 2. Mann-Whitney's U test was performed to analyze difference among groups. * p<0.05, ** p<0.01.

FIGS. 4A, 4B, 4C, 4D, 4E, and 4F are a series of graphs depicting the ACPA fine specificity in the synovial fluid samples of RA patients and their correlation with NI-0101 response. Antibody reactivity against the citrullinated peptides derived from fibrinogen-α (cFbα 556-575; FIG. 4A, FIG. 4D) fibrinogen-β (cFbβ 563-583; FIG. 4B, FIG. 4E) and histone-2A (cH2A 1-20; FIG. 4C, FIG. 4F) were determined by ELISA in synovial fluids from RA patients and correlated with response to NI-0101. FIGS. 4A-4C, RASF samples from ACPA positive RA patients. FIG. 4D-4F, RASF samples from ACPA+ and ACPA− RA patients. RASF samples were classified as NI-0101 non-responders (NR) or NI-0101 responders (R). The difference in OD (delta OD) was calculated as the immunoreactivity against citrulline peptide minus the immunoreactivity against arginine control peptide. The data are shown as arbitrary units where the delta OD for each RASF sample was normalized by the threshold calculated with non-RA SF samples (set at 1, dashed line) above which a sample is considered positive. Mann-Whitney's U test was performed to compare changes observed. *** p<0.001, ** p<0.01, * p<0.05.

FIGS. 5A, 5B, 5C, 5D, 5E, 5F, 5G, and 5H are a series of graphs depicting the ACPA fine specificity in paired sera samples of RA patients and their correlation with RASF response to NI-0101. FIG. 5A depicts the correlation between ACPA levels in paired RA sera and synovial fluids (n=22). FIG. 5B depicts the ACPA levels in paired RA sera classified according to RASF response to NI-0101 (NI-0101 non-responders (NR) or NI-0101 responders (R)). FIGS. 5C-5H) depict antibody reactivity against the citrullinated peptides derived from fibrinogen-α (cFbα 556-575; FIG. 5C, FIG. 5F), fibrinogen-β (cFbβ 563-583; FIG. 5D, FIG. 5G) and histone-2A (cH2A 1-20; FIG. 5E, FIG. 5H) were determined by ELISA in paired sera from RA patients and correlated with response to NI-0101. C-E, Paired sera samples from ACPA positive RA patients (n=10). FIGS. 5F-5H, Paired RA sera samples from ACPA+ and ACPA− RA patients. The difference in OD (delta OD) was calculated as the immunoreactivity against citrulline peptide minus the immunoreactivity against arginine control peptide. The data are shown as arbitrary units where the delta OD for each RA sera sample was normalized by the threshold calculated with non-RA sera samples (set at 1, dashed line) above which a sample is considered positive. Mann-Whitney's U test was performed to compare changes observed. ** p<0.01, * p<0.05.

### DETAILED DESCRIPTION OF THE INVENTION

The compositions and methods provided herein are useful in identifying or otherwise refining a patient population suffering from a TLR4-related disorder, where the patient has an elevated level of anti-citrullinated protein antibodies (ACPA) and/or antibody against specific citrullinated peptides. These patients are identified as suitable candidates for treatment with an agent (e.g., antibodies or other polypeptide-based therapeutics, peptide-based therapeutics, small molecule inhibitors, nucleic acid-based therapeutics and derivatives thereof) that interferes with or otherwise antagonizes TLR4 signaling and neutralizes at least one biological activity of TLR4, alone or in the context of the accessory protein MD-2 as the TLR4/MD-2 complex.

Increased expression of Toll-like receptor 4 (TLR4) and its endogenous ligands have been reported in subgroups of patients with rheumatoid arthritis (RA). However, it is yet to be elucidated whether the increased expression of TLR4 ligands drives inflammation in those patients. The studies presented herein were designed to investigate the effect of specific TLR4 activators present in synovial fluid samples from RA patients (RASF) on RASF-induced proinflammatory cytokine production using primary cells from RA patients.

Briefly, the capacity of RASF to stimulate cytokine production from RA monocytes was analyzed by ELISA. The presence of TLR4 activators in RASF was confirmed by measuring the levels of anti-citrullinated protein antibodies (ACPA), ACPA subtypes with reactivity to specific citrullinated peptides as well as other TLR4 ligands (e.g. HMGB1). Neutralization of TLR4 signaling was investigated using NI-0101, a new therapeutic antibody targeting TLR4. The correlation between TLR4 activators and neutralization was assessed.

RASF from individual RA patients revealed a heterogeneous capacity to induce production of proinflammatory cytokines by monocytes from RA patients. In a subset of RASF, the stimulation was TLR4-dependent, as NI-0101 was able to inhibit the cytokine production. Biomarker analysis demonstrated that TLR4-dependent cytokine induction positively correlated with ACPA positivity and the levels of HMGB1 in the RASF. However, a small group of ACPA+ samples induced cytokines in a TLR4-independent manner. The profiling of ACPA+ RASF as well as paired RA sera samples by their reactivity to different citrullinated peptides identified the TLR4-dependent subgroup with greater specificity.

These studies demonstrate in vitro the contribution of TLR4 to the inflammatory processes in subgroups of RA patients. Using a combination of ACPA and specific citrullinated peptide reactivity, fine profiling is used to identify patients that have a TLR4-driven disease.

In some patients suffering from or suspected of suffering from a disorder, fluids and other biological samples contain elevated levels of TLR4 ligands such as immune complexes containing ACPA and citrullinated proteins and/or peptides. These TLR4 ligands stimulate cells to produce pro-inflammatory cytokines. However, use of an anti-TLR4 antagonist that interferes with or otherwise antagonizes TLR4 signaling, e.g., a neutralizing anti-TLR4 antibody or other anti-TLR4 agent, is shown herein to block this stimulation in patients exhibiting an elevated level of expression for one or more TLR4 ligands and/or other related biomarkers. Thus, the compositions and methods are useful in treating, delaying the progression of or otherwise ameliorating a symptom of a disorder that is dependent on, driven by, or otherwise associated with TLR4 signaling, aberrant, e.g., elevated, TLR4 ligand expression and/or activity, aberrant pro-inflammatory cytokine production, and/or combinations thereof, by administering an anti-TLR4 antagonist, e.g., a neutralizing anti-TLR4 antibody or other polypeptide-based therapeutic, a peptide-based therapeutic, a small molecule inhibitor, a nucleic acid-based therapeutic and derivatives thereof, to patients exhibiting an elevated level of expression for one or more TLR4 ligands and/or related biomarkers. Patients that are likely suitable candidates for treatment with the anti-TLR4 antagonist, e.g., neutralizing anti-TLR4 antibody such as those described herein, are identified by detecting the level of one or more TLR4 ligands or other related biomarkers.

Suitable TLR4 ligands and other related biomarkers for use in identifying likely candidates include ACPA and/or antibody directed against one or more specific citrullinated proteins and/or peptides. In some embodiments, the citrullinated peptide is derived from citrullinated fibrinogen (cFb). In some embodiments, the citrullinated peptide is derived from citrullinated fibrinogen alpha (cFbα). In some embodiments, the citrullinated peptide is derived from citrullinated fibrinogen beta (cFbβ). In some embodiments, the citrullinated peptide is derived from citrullinated histone. In some embodiments, the citrullinated peptide is derived from citrullinated histone 2A.

In some embodiments, the citrullinated peptide comprises the amino acid sequence NTKESSSHHPGIAEFPS-Cit-GK (SEQ ID NO: 1), where Cit=citrulline. This peptide is referred to herein as cFbα 556-575.

In some embodiments, the citrullinated peptide comprises the amino acid sequence HHPGIAEFPS-Cit-GKSSSYSKQF (SEQ ID NO: 2), where Cit=citrulline. This peptide is referred to herein as citFbβ 563-583.

In some embodiments, the citrullinated peptide comprises the amino acid sequence MSG-Cit-GKQGGKA-Cit-AKAKS-Cit-SS (SEQ ID NO: 3), where Cit=citrulline. This peptide is referred to herein as citH2A 1-20.

In some embodiments, ACPA expression levels are detected in conjunction with one or more of the peptides of SEQ ID NO: 1, SEQ ID NO: 2, and/or SEQ ID NO: 3.

In some embodiments, ACPA expression levels are detected in conjunction with the peptides of SEQ ID NO: 2 and the peptide of SEQ ID NO: 3.

The methods provided herein use agents that neutralize TLR4 activity, e.g., TLR4-mediated signaling, and are effective to substantially or completely block pro-inflammatory cytokine production by activated cells in samples from patients suffering from or at risk for a disorder. Anti-TLR4 antagonists are considered to completely block pro-inflammatory cytokine production by activated cells when the level of pro-inflammatory cytokine production by activated cells in the presence of the anti-TLR4 is decreased by at least 95%, e.g., by 96%, 97%, 98%, 99% or 100% as compared to the level of pro-inflammatory cytokine production by activated cells in the absence of interaction, e.g., binding, with the anti-TLR4 antagonist. Anti-TLR4 antagonists are considered to partially block pro-inflammatory cytokine production by activated cells when the level of pro-inflammatory cytokine production by activated cells in the presence of the anti-TLR4 is decreased by at least 50%, e.g., 55%, 60%, 75%, 80%, 85% or 90% as compared to the level of pro-inflammatory cytokine production by activated cells in the absence of interaction, e.g., binding, with the anti-TLR4 antagonist.

Disorders that are useful with the compositions and methods of the invention include any disorder where aberrant, e.g., elevated, TLR4 expression and/or activity, with aberrant TLR4/MD-2 activation and/or aberrant TLR4 ligand activity (e.g., aberrant stimulation of pro-inflammatory cytokine production such as aberrant stimulation of IL-6, TNFα and/or IL-8 production). For example, some TLR4 ligands are believed to be associated with various disorders, such as, by way of non-limiting example, rheumatoid arthritis, osteoarthritis and other arthritic joint diseases, juvenile idiopathic arthritis (JIA), psoriatic arthritis, sepsis, acute lung injury, ischemial reperfusion such as, for example, hepatic and/or cardiac ischemial reperfusion, Type 1 diabetes, islet transplantation, lupus, transplant rejection, atherosclerosis, Sjogren's syndrome, Alzheimer disease, and/or cancer.

Neutralizing anti-TLR4 antibodies of the invention include, for example, the heavy chain complementarity determining regions (CDRs) shown below in Table 2A, the light chain CDRs shown in Table 2B, and combinations thereof.

TABLE 2A
VH CDR sequences from antibody clones
that bind and neutralize TLR4

| Clone ID | Heavy CDR1 | Heavy CDR2 | Heavy CDR3 |
|---|---|---|---|
| Nl-0101 | GGYSWH | YIHYSGYTDFNPSLKT | KDPSDAFPY |
| | (SEQ ID NO: 139) | (SEQ ID NO: 140) | (SEQ ID NO: 141) |
| 1A1 | GYSITGGYS | IHYSGYT | ARKDSGRLLPY |
| | (SEQ ID NO: 15) | (SEQ ID NO: 22) | (SEQ ID NO: 25) |
| 1A6 | GYSITGGYS | IHYSGYT | ARKDSGKWLPY |
| | (SEQ ID NO: 15) | (SEQ ID NO: 22) | (SEQ ID NO: 26) |
| 1812 | GYSITGGYS | IHYSGYT | ARKDSGHLMPY |
| | (SEQ ID NO: 15) | (SEQ ID NO: 22) | (SEQ ID NO: 27) |
| 1C7 | GYSITGGYS | IHYSGYT | ARKDSGHNYPY |
| | (SEQ ID NO: 15) | (SEQ ID NO: 22) | (SEQ ID NO: 28) |
| 1C10 | GYSITGGYS | IHYSGYT | ARKDSGKNFPY |
| | (SEQ ID NO: 15) | (SEQ ID NO: 22) | (SEQ ID NO: 29) |
| 1C12 | GYSITGGYS | IHYSGYT | ARKDSGQLFPY |
| | (SEQ ID NO: 15) | (SEQ ID NO: 22) | (SEQ ID NO: 30) |
| 1010 | GYSITGGYS | IHYSGYT | ARKDSGHNLPY |
| | (SEQ ID NO: 15) | (SEQ ID NO: 22) | (SEQ ID NO: 31) |
| 1E11 | GYSITGGYS | IHYSGYT | ARKDSGNYFPY |
| | (SEQ ID NO: 15) | (SEQ ID NO: 22) | (SEQ ID NO: 24) |
| 1E11 N103D | GYSITGGYS | IHYSGYT | ARKDSGDYFPY |
| | (SEQ ID NO: 15) | (SEQ ID NO: 22) | (SEQ ID NO: 32) |
| 1G12 | GYSITGGYS | IHYSGYT | ARKDSGRYWPY |
| | (SEQ ID NO: 15) | (SEQ ID NO: 22) | (SEQ ID NO: 33) |
| 1E11.C1 | GFPIRYGYS | IHYSGYT | ARKDSGNYFPY |
| | (SEQ ID NO: 16) | (SEQ ID NO: 22) | (SEQ ID NO: 24) |
| 1E11.C2 | GYPIRFGYS | IHYSGYT | ARKDSGNYFPY |
| | (SEQ ID NO: 17) | (SEQ ID NO: 22) | (SEQ ID NO: 24) |
| 1E11.C3 | GYPIRHGYS | IHYSGYT | ARKDSGNYFPY |
| | (SEQ ID NO: 18) | (SEQ ID NO: 22) | (SEQ ID NO: 24) |
| 1E11.C4 | GFPIGQGYS | IHYSGYT | ARKDSGNYFPY |
| | (SEQ ID NO: 19) | (SEQ ID NO: 22) | (SEQ ID NO: 24) |
| 1E11.C5 | GYPIWGGYS | IHYSGYT | ARKDSGNYFPY |
| | (SEQ ID NO: 20) | (SEQ ID NO: 22) | (SEQ ID NO: 24) |
| 1E11.C6 | GYPIGGGYS | IHYSGYT | ARKDSGNYFPY |
| | (SEQ ID NO: 21) | (SEQ ID NO: 22) | (SEQ ID NO: 24) |
| 1E11.E1 | GYSITGGYS | IHYSGYT | ARKDSGNYFPY |
| | (SEQ ID NO: 15) | (SEQ ID NO: 22) | (SEQ ID NO: 24) |
| 1E11.E2 | GYSITGGYS | IHYSGYT | ARKDSGNYFPY |
| | (SEQ ID NO: 15) | (SEQ ID NO: 22) | (SEQ ID NO: 24) |
| 1E11.E3 | GYSITGGYS | IHYSGYT | ARKDSGNYFPY |
| | (SEQ ID NO: 15) | (SEQ ID NO: 22) | (SEQ ID NO: 24) |
| 1E11.E4 | GYSITGGYS | IHYSGYT | ARKDSGNYFPY |
| | (SEQ ID NO: 15) | (SEQ ID NO: 22) | (SEQ ID NO: 24) |
| 1E11.E5 | GYSITGGYS | IHYSGYT | ARKDSGNYFPY |
| | (SEQ ID NO: 15) | (SEQ ID NO: 22) | (SEQ ID NO: 24) |
| 1E11.C2E1 | GYPIRFGYS | IHYSGYT | ARKDSGNYFPY |
| | (SEQ ID NO: 17) | (SEQ ID NO: 22) | (SEQ ID NO: 24) |
| 1E11.C2E3 | GYPIRFGYS | IHYSGYT | ARKDSGNYFPY |
| | (SEQ ID NO: 17) | (SEQ ID NO: 22) | (SEQ ID NO: 24) |
| 1E11.C2E4 | GYPIRFGYS | IHYSGYT | ARKDSGNYFPY |
| | (SEQ ID NO: 17) | (SEQ ID NO: 22) | (SEQ ID NO: 24) |
| 1E11.C2E5 | GYPIRFGYS | IHYSGYT | ARKDSGNYFPY |
| | (SEQ ID NO: 17) | (SEQ ID NO: 22) | (SEQ ID NO: 24) |

TABLE 2B
VL CDR sequences from antibody clones that
bind and neutralize TLR4

| Clone ID | Light CDR1 | Light CDR2 | Light CDR3 |
|---|---|---|---|
| Nl-0101 | RASQSISDHLH | YASHAIS | QQGHSFPLT |
| | (SEQ ID NO: 4) | (SEQ ID NO: 5) | (SEQ ID NO: 6) |
| 1A1 | QSISDH | YAS | QQGHSFPLT |
| | (SEQ ID NO: 34) | (SEQ ID NO: 35) | (SEQ ID NO: 6) |
| 1A6 | QSISDH | YAS | QQGHSFPLT |
| | (SEQ ID NO: 34) | (SEQ ID NO: 35) | (SEQ ID NO: 6) |
| 1812 | QSISDH | YAS | QQGHSFPLT |
| | (SEQ ID NO: 34) | (SEQ ID NO: 35) | (SEQ ID NO: 6) |
| 1C7 | QSISDH | YAS | QQGHSFPLT |
| | (SEQ ID NO: 34) | (SEQ ID NO: 35) | (SEQ ID NO: 6) |
| 1C10 | QSISDH | YAS | QQGHSFPLT |
| | (SEQ ID NO: 34) | (SEQ ID NO: 35) | (SEQ ID NO: 6) |
| 1C12 | QSISDH | YAS | QQGHSFPLT |
| | (SEQ ID NO: 34) | (SEQ ID NO: 35) | (SEQ ID NO: 6) |
| 1010 | QSISDH | YAS | QQGHSFPLT |
| | (SEQ ID NO: 34) | (SEQ ID NO: 35) | (SEQ ID NO: 6) |
| 1E11 | QSISDH | YAS | QQGHSFPLT |
| | (SEQ ID NO: 34) | (SEQ ID NO: 35) | (SEQ ID NO: 6) |
| 1E11 N103D | QSISDH | YAS | QQGHSFPLT |
| | (SEQ ID NO: 34) | (SEQ ID NO: 35) | (SEQ ID NO: 6) |
| 1G12 | QSISDH | YAS | QQGHSFPLT |
| | (SEQ ID NO: 34) | (SEQ ID NO: 35) | (SEQ ID NO: 6) |
| 1E11.C1 | QSISDH | YAS | QQGHSFPLT |
| | (SEQ ID NO: 34) | (SEQ ID NO: 35) | (SEQ ID NO: 6) |
| 1E11.C2 | QSISDH | YAS | QQGHSFPLT |
| | (SEQ ID NO: 34) | (SEQ ID NO: 35) | (SEQ ID NO: 6) |
| 1E11.C3 | QSISDH | YAS | QQGHSFPLT |
| | (SEQ ID NO: 34) | (SEQ ID NO: 35) | (SEQ ID NO: 6) |
| 1E11.C4 | QSISDH | YAS | QQGHSFPLT |
| | (SEQ ID NO: 34) | (SEQ ID NO: 35) | (SEQ ID NO: 6) |
| 1E11.C5 | QSISDH | YAS | QQGHSFPLT |
| | (SEQ ID NO: 34) | (SEQ ID NO: 35) | (SEQ ID NO: 6) |
| 1E11.C6 | QSISDH | YAS | QQGHSFPLT |
| | (SEQ ID NO: 34) | (SEQ ID NO: 35) | (SEQ ID NO: 6) |
| 1E11.E1 | QSISDH | YAS | QQGNDFPVT |
| | (SEQ ID NO: 34) | (SEQ ID NO: 35) | (SEQ ID NO: 37) |
| 1E11.E2 | QSISDH | YAS | QQGYDEPFT |
| | (SEQ ID NO: 34) | (SEQ ID NO: 35) | (SEQ ID NO: 38) |
| 1E11.E3 | QSISDH | YAS | QQGYDFPLT |
| | (SEQ ID NO: 34) | (SEQ ID NO: 35) | (SEQ ID NO: 39) |
| 1E11.E4 | QSISDH | YAS | QQGYDYPLT |
| | (SEQ ID NO: 34) | (SEQ ID NO: 35) | (SEQ ID NO: 40) |
| 1E11.E5 | QSISDH | YAS | QQGYEFPLT |
| | (SEQ ID NO: 34) | (SEQ ID NO: 35) | (SEQ ID NO: 41) |
| 1E11.C2E1 | QSISDH | YAS | QQGNDFPVT |
| | (SEQ ID NO: 34) | (SEQ ID NO: 35) | (SEQ ID NO: 37) |
| 1E11.C2E3 | QSISDH | YAS | QQGYDFPLT |
| | (SEQ ID NO: 34) | (SEQ ID NO: 35) | (SEQ ID NO: 39) |
| 1E11.C2E4 | QSISDH | YAS | QQGYDYPLT |
| | (SEQ ID NO: 34) | (SEQ ID NO: 35) | (SEQ ID NO: 40) |
| 1E11.C2E5 | QSISDH | YAS | QQGYEFPLT |
| | (SEQ ID NO: 34) | (SEQ ID NO: 35) | (SEQ ID NO: 41) |

TLR4 antibodies of the invention include, for example, antibodies having the combination of heavy chain and light chain sequences shown below.

Exemplary antibodies of the invention include, for example, the anti-TLR4 antibodies described in PCT/IB2005/004206, filed Jun. 14, 2005 and published as WO 2007/110678, the anti-TLR4 antibodies described in PCT application PCT/IB2008/003978, filed May 14, 2008 and published as WO 2009/101479, the contents of each of which are hereby incorporated by reference in their entirety, and commercially available antibodies such as HTA125.

Exemplary antibodies of the invention include, for example, the antibody referred to herein as NI-0101, which is also referred to herein and in the Figures as “hu15C1,” which binds the human TLR4/MD2 complex and also binds TLR4 independently of the presence of MD-2. The sequences of the N1-0101 (hu15c1) antibody are shown below, with the CDR sequences underlined in the VH and VL amino acid sequences:

### NI-0101 Heavy Chain Nucleotide Sequence:

(SEQ ID NO: 11)

ATGGGATGGAGCTGGATCTTTCTCTTCCTCCTGTCAGGAACTGCAGGTGT
ACATTGCCAGGTGCAGCTTCAGGAGTCCGGCCCAGGACTGGTGAAGCCTT
CGGACACCCTGTCCCTCACCTGCGCTGTCTCTGGTTACTCCATCACCGGT
GGTTATAGCTGGCACTGGATACGGCAGCCCCCAGGGAAGGGACTGGAGTG
GATGGGGTATATCCACTACAGTGGTTACACTGACTTCAACCCCTCCCTCA
AGACTCGAATCACCATATCACGTGACACGTCCAAGAACCAGTTCTCCCTG
AAGCTGAGCTCTGTGACCGCTGTGGACACTGCAGTGTATTACTGTGCGAG
AAAAGATCCGTCCGACGCCTTTCCTTACTGGGGCCAAGGGACTCTGGTCA
CTGTCTCTTCCGCCTCCACCAAGGGCCCATCGGTCTTCCCCCTGGCACCC
TCCTCCAAGAGCACCTCTGGGGGCACAGCGGCCCTGGGCTGCCTGGTCAA
GGACTACTTCCCCGAACCGGTGACGGTGTCGTGGAACTCAGGCGCCCTGA
CCAGCGGCGTGCACACCTTCCCGGCTGTCCTACAGTCCTCAGGACTCTAC
TCCCTCAGCAGCGTGGTGACCGTGCCCTCCAGCAGCTTGGGCACCCAGAC
CTACATCTGCAACGTGAATCACAAGCCCAGCAACACCAAGGTGGACAAGA
GAGTTGAGCCCAAATCTTGTGACAAAACTCACACATGCCCACCGTGCCCA
GCACCTGAACTCCTGGGGGGACCGTCAGTCTTCCTCTTCCCCCCAAAACC
CAAGGACACCCTCATGATCTCCCGGACCCCTGAGGTCACATGCGTGGTGG
TGGACGTGAGCCACGAAGACCCTGAGGTCAAGTTCAACTGGTACGTGGAC
GGCGTGGAGGTGCATAATGCCAAGACAAAGCCGCGGGAGGAGCAGTACAA
CAGCACGTACCGTGTGGTCAGCGTCCTCACCGTCCTGCACCAGGACTGGC
TGAATGGCAAGGAGTACAAATGCAAGGTCTCCAGTAAAGCTTTCCCTGCC
CCCATCGAGAAAACCATCTCCAAAGCCAAAGGGCAGCCCCGAGAACCACA
GGTGTACACCCTGCCCCCATCCCGGGAGGAGATGACCAAGAACCAGGTCA
GCCTGACCTGCCTGGTCAAAGGCTTCTATCCCAGCGACATCGCCGTGGAG
TGGGAGAGCAATGGGCAGCCGGAGAACAACTACAAGACCACGCCTCCCGT
GCTGGACTCCGACGGCTCCTTCTTCCTCTATAGCAAGCTCACCGTGGACA
AGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAA
ATAG

### NI-0101 Heavy Chain Amino Acid Sequence:

(SEQ ID NO: 9)

MGWSWIFLFLLSGTAGVHCQVQLQESGPGLVKPSDTLSLTCAVSGYSITG
GYSWHWIRQPPGKGLEWMGYIHYSGYTDFNPSLKTRITISRDTSKNQFSL
KLSSVTAVDTAVYYCARKDPSDAFPYWGQGTLVTVSSASTKGPSVFPLAP
SSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCP
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSSKAFPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

### NI-0101 Light Chain Nucleotide Sequence:

(SEQ ID NO: 12)

ATGGAATGGAGCTGGGTCTTTCTCTTCTTCCTGTCAGTAACTACAGGTGT
CCACTCCGAAATTGTGTTGACGCAGTCTCCAGACTTTCAGTCTGTGACTC
CAAAGGAAAAAGTCACCATCACCTGCAGGGCCAGTCAGAGTATCAGCGAC
CACTTACACTGGTACCAACAGAAACCTGATCAGTCTCCCAAGCTCCTCAT
CAAATATGCTTCCCATGCCATTTCTGGGGTCCCATCGAGGTTCAGTGGCA
GTGGGTCTGGGACAGACTTCACTCTCACCATCAATAGCCTAGAGGCTGAA
GATGCTGCAACGTATTACTGTCAGCAGGGTCACAGTTTTCCGCTCACTTT
CGGCGGAGGGACCAAGGTGGAGATCAAACGTACGGTGGCTGCACCATCTG
TCTTCATCTTCCCGCCATCTGATGAGCAGTTGAAATCTGGAACTGCCTCT
GTTGTGTGCCTGCTGAATAACTTCTATCCCAGAGAGGCCAAAGTACAGTG
GAAGGTGGATAACGCCCTCCAATCGGGTAACTCCCAGGAGAGTGTCACAG
AGCAGGACAGCAAGGACAGCACCTACAGCCTCAGCAGCACCCTGACGCTG
AGCAAAGCAGACTACGAGAAACACAAAGTCTACGCCTGCGAAGTCACCCA
TCAGGGCCTGAGCTCGCCCGTCACAAAGAGCTTCAACAGGGGAGAGTGTT
AG

### NI-0101 Light Chain Amino Acid Sequence:

(SEQ ID NO: 10)

MEWSWVFLFFLSVTTGVHSEIVLTQSPDFQSVTPKEKVTITCRASQSISD
HLHWYQQKPDQSPKLLIKYASHAISGVPSRFSGSGSGTDFTLTINSLEAE
DAATYYCQQGHSFPLTFGGGTKVEIKRTVAAPSVFIFPPSDEQLKSGTAS
VVCLLNNFYPREAKVQWKVDNALQSGNSQESVTEQDSKDSTYSLSSTLTL
SKADYEKHKVYACEVTHQGLSSPVTKSFNRGEC

The NI-0101 (hu15c1) antibody includes VH CDRs having the sequences GGYSWH (SEQ ID NO: 139), YIHYSGYTDFNPSLKT (SEQ ID NO: 140), and KDPSDAFPY (SEQ ID NO: 141), and VL CDRs having the sequences RASQSISDHLH (SEQ ID NO: 4), YASHAIS (SEQ ID NO: 5) and QQGHSFPLT (SEQ ID NO: 6).

The amino acid and nucleic acid sequences of the heavy chain variable (VH) and light chain variable (VL) regions of the anti-TLR4/MD2 antibodies are shown below. The amino acids encompassing the complementarity determining regions (CDR) as defined by Chothia et al. 1989, E. A. Kabat et al., 1991 are highlighted in underlined and italicized text below. (See Chothia, C, et al., Nature 342:877-883 (1989); Kabat, E A, et al., Sequences of Protein of immunological interest, Fifth Edition, US Department of Health and Human Services, US Government Printing Office (1991)).

Anti-TLR4 antibodies include the antibodies described in U.S. Pat. No. 7,312,320, filed Dec. 10, 2004 and U.S. Pat. No. 7,674,884, filed Jun. 14, 2005 and in WO 05/065015, filed Dec. 10, 2004 and 2007/110678, filed Jun. 14, 2005, each of which is hereby incorporated by reference in its entirety. Several exemplary antibodies include the antibodies referred to therein as 18H10, 1607, 15C1 and 7E3.

The sequences of several exemplary antibodies are shown below.

### 15C1 Hu V_{H} Version 4-28

(SEQ ID NO: 42)

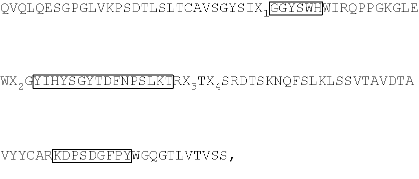

where X₁ is Thr or Ser; X₂ is Ile or Met; X₃ is
Val or Ile; and X₄ is Met or Ile
(SEQ ID NO: 139)
CDR 1: GGYSWH
(SEQ ID NO: 140)
CDR 2: YIHYSGYTDFNPSLKT
(SEQ ID NO: 137)
CDR 3: KDPSDGFPY

### 15C1 Hu V_{H} Version 3-66

(SEQ ID NO: 43)

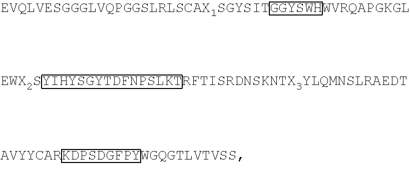

where X₁ is Ala or Val; X₂ is Val or Met; and X₃
is Leu or Phe.
(SEQ ID NO: 139)
CDR 1: GGYSWH
(SEQ ID NO: 140)
CDR 2: YIHYSGYTDFNPSLKT
(SEQ ID NO: 137)
CDR 3: KDPSDGFPY

### 15C1 Hu VL Version L6

(SEQ ID NO: 44)

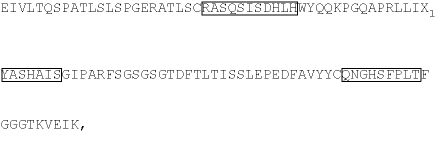

where X₁ is Lys or Tyr.
(SEQ ID NO: 4)
CDR1: RASQSISDHLH
(SEQ ID NO: 5)
CDR2: YASHAIS
(SEQ ID NO: 138)
CDR3: QNGHSFPLT

### 15C1 Hu VL Version A26

(SEQ ID NO: 45)

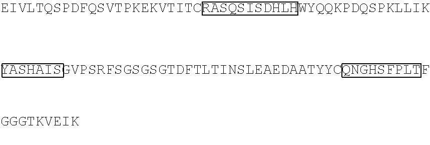

(SEQ ID NO: 4)
CDR1: RASQSISDHLH
(SEQ ID NO: 5)
CDR2: YASHAIS
(SEQ ID NO: 138)
CDR3: QNGHSFPLT

### 18H10 Hu VH Version 1-69

(SEQ ID NO: 46)

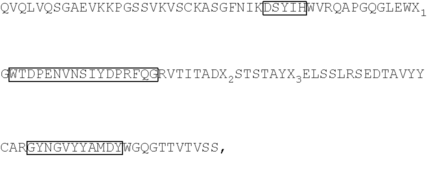

where X₁ is Met or Ile; X₂ is Lys or Thr; and X₃ is
Met or Leu.
(SEQ ID NO: 47)
CDR1: DSYIH
(SEQ ID NO: 48)
CDR2: WTDPENVNSIYDPRFQG
(SEQ ID NO: 49)
CDR3: GYNGVYYAMDY

### 18H10 Hu VL Version L6

(SEQ ID NO: 50)

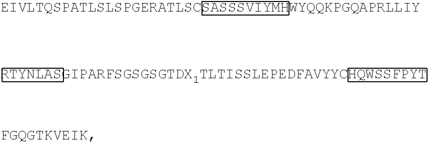

where X₁ is Phe or Tyr.
(SEQ ID NO: 51)
CDR1: SASSSVIYMH
(SEQ ID NO: 52)
CDR2: RTYNLAS
(SEQ ID NO: 53)
CDR3: HQWSSFPYT

### 7E3 Hu VH Version 2-70

(SEQ ID NO: 54)

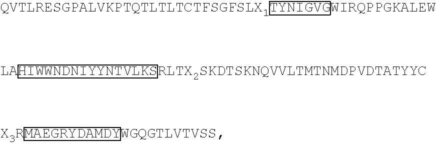

where X₁ is Ser or Thr; X₂ is Ile or Phe; and X₃ is
Ile or Ala.
(SEQ ID NO: 55)
CDR1: TYNIGVG
(SEQ ID NO: 56)
CDR2: HIWWNDNIYYNTVLKS
(SEQ ID NO: 57)
CDR3: MAEGRYDAMDY

### 7E3 Hu VH Version 3-66

(SEQ ID NO: 58)

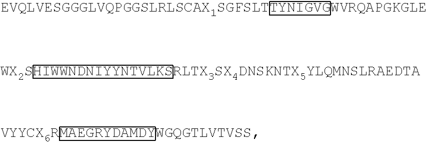

where X₁ is Phe or Ala; X₂ is Val or Leu; X₃ is Ile
or Phe; X₄ is Lys or Arg; X₅ is Leu or Val; and X₆
is Ile or Ala.
(SEQ ID NO: 59)
CDR1: TYNIGVG
(SEQ ID NO: 60)
CDR2: HIWWNDNIYYNTVLKS
(SEQ ID NO: 61)
CDR3: MAEGRYDAMDY

### 7E3 Hu VL Version L19

(SEQ ID NO: 62)

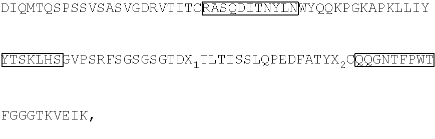

where X₁ is Phe or Tyr; and X₂ is Tyr or Phe.
(SEQ ID NO: 63)
CDR1: RASQDITNYLN
(SEQ ID NO: 64)
CDR2: YTSKLHS
(SEQ ID NO: 65)
CDR3: QQGNTFPWT

Anti-TLR4 antibodies include the antibodies described in PCT/IB2008/003978, filed May 14, 2008 (PCT Publication No. WO 2009/101479), the contents of which are hereby incorporated by reference in their entirety. These anti-TLR4 antibodies are modified to include one or more mutations in the CDR3 portion. The sequences of several exemplary antibodies are shown below.

### 15C1 Humanized VH Mutant 1 Amino Acid Sequence:

(SEQ ID NO: 7)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
PSDAFPYWGQGTLVTVSS

### 15C1 Humanized VH Mutant 1 Nucleic Acid Sequence:

(SEQ ID NO: 66)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
CCGTCCGACGCCTTTCCTTACTGGGGCCAAGGGACTCTGGTCACTGTCTC
TTCC

### 15C1 Humanized VH Mutant 2 Amino Acid Sequence:

(SEQ ID NO: 67)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
PSEGFPYWGQGTLVTVSS

### 15C1 Humanized VH Mutant 2 Nucleic Acid Sequence:

(SEQ ID NO: 68)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
CCGTCCGAGGGATTTCCTTACTGGGGCCAAGGGACTCTGGTCACTGTCTC
TTCC

### 15C1 Humanized VL Mutant 1 Amino Acid Sequence:

(SEQ ID NO: 69)

EIVLTQSPDFQSVTPKEKVTITCRASQSISDHLHWYQQKPDQSPKLLIKY
ASHAISGVPSRFSGSGSGTDFTLTINSLEAEDAATYYCQNSHSFPLTFGG
GTKVEIK

### 15C1 Humanized VL Mutant 1 Nucleic Acid Sequence:

(SEQ ID NO: 70)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGAATAGTCACAGTTTTCCGCTCACTTTCGGCGGA
GGGACCAAGGTGGAGATCAAA

### 15C1 Humanized VL Mutant 2 Amino Acid Sequence:

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

### 15C1 Humanized VL Mutant 2 Nucleic Acid Sequence:

(SEQ ID NO: 71)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 15C1 Humanized VL Mutant 3 Amino Acid Sequence:

(SEQ ID NO: 72)

EIVLTQSPDFQSVTPKEKVTITCRASQSISDHLHWYQQKPDQSPKLLIKY
ASHAISGVPSRFSGSGSGTDFTLTINSLEAEDAATYYCQNSSSFPLTFGG
GTKVEIK

### 15C1 Humanized VL Mutant 3 Nucleic Acid Sequence:

(SEQ ID NO: 73)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGAATAGTAGTAGTTTTCCGCTCACTTTCGGCGGA
GGGACCAAGGTGGAGATCAAA

### 15C1 Humanized VL Mutant 4 Amino Acid Sequence:

(SEQ ID NO: 74)

EIVLTQSPDFQSVTPKEKVTITCRASQSISDHLHWYQQKPDQSPKLLIKY
ASHAISGVPSRFSGSGSGTDFTLTINSLEAEDAATYYCQQSHSFPLTFGG
GTKVEIK

### 15C1 Humanized VL Mutant 4 Nucleic Acid Sequence:

(SEQ ID NO: 75)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGAGTCACAGTTTTCCGCTCACTTTCGGCGGA
GGGACCAAGGTGGAGATCAAA

Antibodies of the invention interfere with or otherwise antagonize signaling via human and/or cynomolgus monkey TLR4 and/or human and/or cynomolgus monkey TLR4/MD-2 complexes. In some embodiments, the antibody binds to an epitope that includes one or more amino acid residues on human and/or cynomolgus monkey TLR4 having the following sequences:

### Human TLR4 Amino Acid Sequence

(SEQ ID NO: 76)

MMSASRLAGTLIPAMAFLSCVRPESWEPCVEVVPNITYQCMELNFYKIPD
NLPFSTKNLDLSFNPLRHLGSYSFFSFPELQVLDLSRCEIQTIEDGAYQS
LSHLSTLILTGNPIQSLALGAFSGLSSLQKLVAVETNLASLENFPIGHLK
TLKELNVAHNLIQSFKLPEYFSNLTNLEHLDLSSNKIQSIYCTDLRVLHQ
MPLLNLSLDLSLNPMNFIQPGAFKEIRLHKLTLRNNFDSLNVMKTCIQGL
AGLEVHRLVLGEFRNEGNLEKFDKSALEGLCNLTIEEFRLAYLDYYLDDI
IDLFNCLTNVSSFSLVSVTIERVKDFSYNFGWQHLELVNCKFGQFPTLKL
KSLKRLTFTSNKGGNAFSEVDLPSLEFLDLSRNGLSFKGCCSQSDFGTTS
LKYLDLSFNGVITMSSNFLGLEQLEHLDFQHSNLKQMSEFSVFLSLRNLI
YLDISHTHTRVAFNGIFNGLSSLEVLKMAGNSFQENFLPDIFTELRNLTF
LDLSQCQLEQLSPTAFNSLSSLQVLNMSHNNFFSLDTFPYKCLNSLQVLD
YSLNHIMTSKKQELQHFPSSLAFLNLTQNDFACTCEHQSFLQWIKDQRQL
LVEVERMECATPSDKQGMPVLSLNITCQMNKTIIGVSVLSVLVVSVVAVL
VYKFYFHLMLLAGCIKYGRGENIYDAFVIYSSQDEDWVRNELVKNLEEGV
PPFQLCLHYRDFIPGVAIAANIIHEGFHKSRKVIVVVSQHFIQSRWCIFE
YEIAQTWQFLSSRAGIIFIVLQKVEKTLLRQQVELYRLLSRNTYLEWEDS
VLGRHIFWRRLRKALLDGKSWNPEGTVGTGCNWQEATSI

### Cynomolgus Monkey TLR4 Amino Acid Sequence 1

(SEQ ID NO: 77)

MTSALRLAGTLIPAMAFLSCVRPESWEPCVEVVPNITYQCMELKFYKIPD
NIPFSTKNLDLSFNPLRHLGSYSFLRFPELQVLDLSRCEIQTIEDGAYQS
LSHLSTLILTGNPIQSLALGAFSGLSSLQKLVAVETNLASLENFPIGHLK
TLKELNVAHNLIQSFKLPEYFSNLTNLEHLDLSSNKIQNIYCKDLQVLHQ
MPLSNLSLDLSLNPINFIQPGAFKEIRLHKLTLRSNFDDLNVMKTCIQGL
AGLEVHRLVLGEFRNERNLEEFDKSSLEGLCNLTIEEFRLTYLDCYLDNI
IDLFNCLANVSSFSLVSVNIKRVEDFSYNFRWQHLELVNCKFEQFPTLEL
KSLKRLTFTANKGGNAFSEVDLPSLEFLDLSRNGLSFKGCCSQSDFGTTS
LKYLDLSFNDVITMSSNFLGLEQLEHLDFQHSNLKQMSQFSVFLSLRNLI
YLDISHTHTRVAFNGIFDGLLSLKVLKMAGNSFQENFLPDIFTDLKNLTF
LDLSQCQLEQLSPTAFDTLNKLQVLNMSHNNFFSLDTFPYKCLPSLQVLD
YSLNHIMTSNNQELQHFPSSLAFLNLTQNDFACTCEHQSFLQWIKDQRQL
LVEAERMECATPSDKQGMPVLSLNITCQMNKTIIGVSVFSVLVVSVVAVL
VYKFYFHLMLLAGCIKYGRGENIYDAFVIYSSQDEDWVRNELVKNLEEGV
PPFQLCLHYRDFIPGVAIAANIIHEGFHKSRKVIVVVSQHFIQSRWCIFE
YEIAQTWQFLSSRAGIIFIVLQKVEKTLLRQQVELYRLLSRNTYLEWEDS
VLGQHIFWRRLRKALLDGKSWNPEEQ

Antibodies of the invention interfere with or otherwise antagonize signaling via human and/or cynomolgus monkey TLR4 and/or human and/or cynomolgus monkey TLR4/MD-2 complexes. In some embodiments, the antibody binds to an epitope that includes one or more amino acid residues on human and/or cynomolgus monkey TLR4 between residues 289 and 375 of SEQ ID NO: 76 (human TLR4) and/or SEQ ID NO: 77 (cynomolgus TLR4). For example, TLR4 antibodies specifically bind to an epitope that includes residue 349 of SEQ ID NO: 76 (human) and/or SEQ ID NO: 77 (cynomolgus). In some embodiments, the epitope also includes additional residues, for example, residues selected from the group consisting of at least residues 328 and 329 of SEQ ID NO: 76 (human) and/or SEQ ID NO: 77 (cynomolgus); at least residue 351 of SEQ ID NO: 76 (human) and/or SEQ ID NO: 77 (cynomolgus); and at least residues 369 through 371 of SEQ ID NO: 76 (human) and/or SEQ ID NO: 77 (cynomolgus), and any combination thereof.

In some embodiments, the invention provides an isolated antibody that specifically binds Toll-like receptor 4 (TLR4), wherein the antibody binds to an epitope that includes at least residue 349 of SEQ ID NO: 76 and an epitope that includes at least residue 349 of SEQ ID NO; 76. In some embodiments, the antibody includes a heavy chain with three complementarity determining regions (CDRs) including a variable heavy chain complementarity determining region 1 (CDRH1) amino acid sequence of GYSITGGYS (SEQ ID NO: 15); a variable heavy chain complementarity determining region 2 (CDRH2) amino acid sequence of IHYSGYT (SEQ ID NO: 22); and a variable heavy chain complementarity determining region 3 (CDRH3) amino acid sequence of ARKDSG(X₁)(X₂)(X₃)PY (SEQ ID NO: 14), where X₁ is N, Q, D or E, X₂ is any hydrophobic amino acid, and X₃ is any hydrophobic amino acid; and a light chain with three CDRs including a variable light chain complementarity determining region 1 (CDRL1) amino acid sequence of QSISDH (SEQ ID NO: 34); a variable light chain complementarity determining region 2 (CDRL2) amino acid sequence of YAS (SEQ ID NO: 35); and a variable light chain complementarity determining region 3 (CDRL3) amino acid sequence of QQGHSFPLT (SEQ ID NO: 6). In some embodiments, the epitope further includes at least residues 328 and 329 of SEQ ID NO: 76 and SEQ ID NO: 76. In some embodiments, the epitope further includes at least residue 351 of SEQ ID NO: 76 and SEQ ID NO: 76. In some embodiments, the epitope further includes one or more residues between residues 369 through 371 of SEQ ID NO: 76 and SEQ ID NO: 76. In some embodiments, the epitope further includes at least residues 369 through 371 of SEQ ID NO: 76 and SEQ ID NO: 76. In some embodiments, the antibody specifically binds to an epitope that includes at least residues 328, 329, 349, 351 and 369 through 371 of SEQ ID NO: 76 and SEQ ID NO: 76. In some embodiments, the antibody further includes an amino acid substitution in the gamma heavy chain constant region at EU amino acid position 325 and an amino acid substitution at EU amino acid position 328. In some embodiments, the amino acid substituted at EU amino acid position 325 is serine, and wherein the amino acid substituted at EU amino acid position 328 is phenylalanine.

An exemplary TLR4 monoclonal antibody is the 1E11 antibody described herein. As shown below, the 1E11 antibody includes a heavy chain variable region (SEQ ID NO: 78) encoded by the nucleic acid sequence shown in SEQ ID NO: 79, and a light chain variable region (SEQ ID NO: 8) encoded by the nucleic acid sequence shown in SEQ ID NO: 80.

### 1E11 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1A1 antibody described herein. As shown below, the 1A1 antibody includes a heavy chain variable region (SEQ ID NO: 82) encoded by the nucleic acid sequence shown in SEQ ID NO: 81, and a light chain variable region (SEQ ID NO: 8) encoded by the nucleic acid sequence shown in SEQ ID NO: 80.

### 1A1 VH Nucleic Acid Sequence

(SEQ ID NO: 81)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGCCGCCTCCTCCCTTACTGGGGCCAAGGGACTCTGGTCACTGTCTC
TTCC

### 1A1 VH Amino Acid Sequence

(SEQ ID NO: 82)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGRLLPYWGQGTLVTVSS

### 1A1 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1A1 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1A1 antibody have the following sequences: GYSITGGYS (SEQ ID NO: 15); IHYSGYT (SEQ ID NO: 22); and ARKDSGRLLPY (SEQ ID NO: 25). The light chain CDRs of the 1A1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1A6 antibody described herein. As shown below, the 1A6 antibody includes a heavy chain variable region (SEQ ID NO: 84) encoded by the nucleic acid sequence shown in SEQ ID NO: 83, and a light chain variable region (SEQ ID NO: 8) encoded by the nucleic acid sequence shown in SEQ ID NO: 80.

### 1A6 VH Nucleic Acid Sequence

(SEQ ID NO: 83)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCAAGTGGTTGCCTTACTGGGGCCAAGGGACTCTGGTCACTGTCTC
TTCC

### 1A6 VH Amino Acid Sequence

(SEQ ID NO: 84)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGKWLPYWGQGTLVTVSS

### 1A6 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1A6 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.IP.37 (2000) LIGM:230). The heavy chain CDRs of the 1A6 antibody have the following sequences: GYSITGGYS (SEQ ID NO: 15); IHYSGYT (SEQ ID NO: 22); and ARKDSGKWLPY (SEQ ID NO: 26). The light chain CDRs of the 1A6 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1B12 antibody described herein. As shown below, the 1B12 antibody includes a heavy chain variable region (SEQ ID NO: 86) encoded by the nucleic acid sequence shown in SEQ ID NO: 85, and a light chain variable region (SEQ ID NO: 8) encoded by the nucleic acid sequence shown in SEQ ID NO: 80.

### 1B12 VH Nucleic Acid Sequence

(SEQ ID NO: 85)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGGCACCTCATGCCTTACTGGGGCCAAGGGACTCTGGTCACTGTCTC
TTCC

### 1B12 VH Amino Acid Sequence

(SEQ ID NO: 86)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHLMPYWGQGTLVTVSS

### 1B12 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1B12 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1A6 antibody have the following sequences: GYSITGGYS (SEQ ID NO: 15); IHYSGYT (SEQ ID NO: 22); and ARKDSGHLMPY (SEQ ID NO: 27). The light chain CDRs of the 1B12 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1C7 antibody described herein. As shown below, the 1C7 antibody includes a heavy chain variable region (SEQ ID NO: 88) encoded by the nucleic acid sequence shown in SEQ ID NO: 87, and a light chain variable region (SEQ ID NO: 8) encoded by the nucleic acid sequence shown in SEQ ID NO: 80.

### 1C7 VH Nucleic Acid Sequence

(SEQ ID NO: 87)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGGCACAACTACCCTTACTGGGGCCAAGGGACTCTGGTCACTGTCTC
TTCC

### 1 C7 VH Amino Acid Sequence

(SEQ ID NO: 88)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHNYPYWGQGTLVTVSS

### 1 C7 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1 C7 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C7 antibody have the following sequences: GYSITGGYS (SEQ ID NO: 15); IHYSGYT (SEQ ID NO: 22); and ARKDSGHNYPY (SEQ ID NO: 28). The light chain CDRs of the 1C7 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1C10 antibody described herein. As shown below, the 1C10 antibody includes a heavy chain variable region (SEQ ID NO: 90) encoded by the nucleic acid sequence shown in SEQ ID NO: 89, and a light chain variable region (SEQ ID NO: 8) encoded by the nucleic acid sequence shown in SEQ ID NO: 80.

### 1C10 VH Nucleic Acid Sequence

(SEQ ID NO: 89)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCAAGAACTTCCCTTACTGGGGCCAAGGGACTCTGGTCACTGTCTC
TTCC

### 1C10 VH Amino Acid Sequence

(SEQ ID NO: 90)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGKNFPYWGQGTLVTVSS

### 1C10 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1C10 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C10 antibody have the following sequences: GYSITGGYS (SEQ ID NO: 15); IHYSGYT (SEQ ID NO: 22); and ARKDSGKNFPY (SEQ ID NO: 29). The light chain CDRs of the 1C10 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1C12 antibody described herein. As shown below, the 1C12 antibody includes a heavy chain variable region (SEQ ID NO: 92) encoded by the nucleic acid sequence shown in SEQ ID NO: 91, and a light chain variable region (SEQ ID NO: 8) encoded by the nucleic acid sequence shown in SEQ ID NO: 80.

### 1C12 VH Nucleic Acid Sequence

(SEQ ID NO: 91)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCCAGTTGTTCCCTTACTGGGGCCAAGGGACTCTGGTCACTGTCTC
TTCC

### 1C12 VH Amino Acid Sequence

(SEQ ID NO: 92)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGQLFPYWGQGTLVTVSS

### 1C12 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1C12 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1C12 antibody have the following sequences: GYSITGGYS (SEQ ID NO: 15); IHYSGYT (SEQ ID NO: 22); and ARKDSGQLFPY (SEQ ID NO: 30). The light chain CDRs of the 1C12 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1D10 antibody described herein. As shown below, the 1D10 antibody includes a heavy chain variable region (SEQ ID NO: 94) encoded by the nucleic acid sequence shown in SEQ ID NO: 93, and a light chain variable region (SEQ ID NO: 8) encoded by the nucleic acid sequence shown in SEQ ID NO: 80.

### 1D10 VH Nucleic Acid Sequence

(SEQ ID NO: 93)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
AGCGGCCACAACTTGCCTTACTGGGGCCAAGGGACTCTGGTCACTGTCTC
TTcc

### 1D10 VH Amino Acid Sequence

(SEQ ID NO: 94)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGHNLPYWGQGTLVTVSS

### 1D10 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1D10 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.IP.37 (2000) LIGM:230). The heavy chain CDRs of the 1D10 antibody have the following sequences: GYSITGGYS (SEQ ID NO: 15); IHYSGYT (SEQ ID NO: 22); and ARKDSGHNLPY (SEQ ID NO: 31). The light chain CDRs of the 1D10 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11 N103D antibody described herein. As shown below, the 1E11 N103D antibody includes a heavy chain variable region (SEQ ID NO: 96) encoded by the nucleic acid sequence shown in SEQ ID NO: 95, and a light chain variable region (SEQ ID NO: 8) encoded by the nucleic acid sequence shown in SEQ ID NO: 80.

### 1E11 N103D VH Nucleic Acid Sequence

(SEQ ID NO: 95)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCGACTACTTCCCTTACTGGGGCCAAGGGACTCTGGTCACTGTCTC
TTCC

### 1E11 N103D VH Amino Acid Sequence

(SEQ ID NO: 96)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGDYFPYWGQGTLVTVSS

### 1E11 N103D VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11 N103D VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11 N103D antibody have the following sequences: GYSITGGYS (SEQ ID NO: 15); IHYSGYT (SEQ ID NO: 22); and ARKDSGDYFPY (SEQ ID NO: 32). The light chain CDRs of the 1E11 N103D antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1G12 antibody described herein. As shown below, the 1012 antibody includes a heavy chain variable region (SEQ ID NO: 98) encoded by the nucleic acid sequence shown in SEQ ID NO: 97, and a light chain variable region (SEQ ID NO: 8) encoded by the nucleic acid sequence shown in SEQ ID NO: 80.

### 1G12 VH Nucleic Acid Sequence

(SEQ ID NO: 97)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCCGGGCGGTACTGGCCTTACTGGGGCCAAGGGACTCTGGTCACTGTCTC
TTCC

### 1G12 VH Amino Acid Sequence

(SEQ ID NO: 98)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGRYWPYWGQGTLVTVSS

### 1G12 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1G12 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1012 antibody have the following sequences: GYSITGGYS (SEQ ID NO: 15); IHYSGYT (SEQ ID NO: 22); and ARKDSGRYWPY (SEQ ID NO: 33). The light chain CDRs of the 1E11 N103D antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C1 antibody described herein. As shown below, the 1E11.C1 antibody includes a heavy chain variable region (SEQ ID NO: 100) encoded by the nucleic acid sequence shown in SEQ ID NO: 99, and a light chain variable region (SEQ ID NO: 8) encoded by the nucleic acid sequence shown in SEQ ID NO: 80.

### 1E11.C1 VH Nucleic Acid Sequence

(SEQ ID NO: 99)

CAGGTGCAGCTTCAGGAGTCCGGCCCAGGACTGGTGAAGCCTTCGGACAC
CCTGTCCCTCACCTGCGCTGTCTCTGGTTTCCCGATCCGCTACGGGTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C1 VH Amino Acid Sequence

(SEQ ID NO: 100)

QVQLQESGPGLVKPSDTLSLTCAVSGFPIRYGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C1 VL Amino Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C1 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C1 antibody have the following sequences: GFPIRYGYS (SEQ ID NO: 16); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C2 antibody described herein. As shown below, the 1E11.C2 antibody includes a heavy chain variable region (SEQ ID NO: 102) encoded by the nucleic acid sequence shown in SEQ ID NO: 101, and a light chain variable region (SEQ ID NO: 80) encoded by the nucleic acid sequence shown in SEQ ID NO: 8.

### 1E11.C2 VH Nucleic Acid Sequence

(SEQ ID NO: 101)

CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C2 VH Amino Acid Sequence

(SEQ ID NO: 102)

QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C2 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C2 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2 antibody have the following sequences: GYPIRFGYS (SEQ ID NO: 17); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C3 antibody described herein. As shown below, the 1E11.C3 antibody includes a heavy chain variable region (SEQ ID NO: 104) encoded by the nucleic acid sequence shown in SEQ ID NO: 103, and a light chain variable region (SEQ ID NO: 8) encoded by the nucleic acid sequence shown in SEQ ID NO: 80.

### 1E11.C3 VH Nucleic Acid Sequence

(SEQ ID NO: 103)

CAGGTGCAGCTTCAGGAGTCCGGCCCAGGACTGGTGAAGCCTTCGGACAC
CCTGTCCCTCACCTGCGCTGTCTCTGGTTACCCCATCCGGCACGGGTACA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C3 VH Amino Acid Sequence

(SEQ ID NO: 104)

QVQLQESGPGLVKPSDTLSLTCAVSGYPIRHGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C3 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C3 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C3 antibody have the following sequences: GYPIRHGYS (SEQ ID NO: 18); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C4 antibody described herein. As shown below, the 1E11.C4 antibody includes a heavy chain variable region (SEQ ID NO: 106) encoded by the nucleic acid sequence shown in SEQ ID NO: 105, and a light chain variable region (SEQ ID NO: 8) encoded by the nucleic acid sequence shown in SEQ ID NO: 80.

### 1E11.C4 VH Nucleic Acid Sequence

(SEQ ID NO: 105)

CAGGTGCAGCTTCAGGAGTCCGGCCCAGGACTGGTGAAGCCTTCGGACAC
CCTGTCCCTCACCTGCGCTGTCTCTGGTTTCCCGATCGGCCAGGGGTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1 E11.C4 VH Amino Acid Sequence

(SEQ ID NO: 106)

QVQLQESGPGLVKPSDTLSLTCAVSGFPIGQGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C4 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C4 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C4 antibody have the following sequences: GFPIGQGYS (SEQ ID NO: 19); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C5 antibody described herein. As shown below, the 1E11.C5 antibody includes a heavy chain variable region (SEQ ID NO: 108) encoded by the nucleic acid sequence shown in SEQ ID NO: 107, and a light chain variable region (SEQ ID NO: 8) encoded by the nucleic acid sequence shown in SEQ ID NO: 80.

### 1E11.C5 VH Nucleic Acid Sequence

(SEQ ID NO: 107)

CAGGTGCAGCTTCAGGAGTCCGGCCCAGGACTGGTGAAGCCTTCGGACAC
CCTGTCCCTCACCTGCGCTGTCTCTGGTTACCCGATCTGGGGGGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCCGCCTCCACC

### 1E11.C5 VH Amino Acid Sequence

(SEQ ID NO: 108)

QVQLQESGPGLVKPSDTLSLTCAVSGYPIWGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C5 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C5 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.IP.37 (2000) LIGM:230). The heavy chain CDRs of the 1E11.C5 antibody have the following sequences: GYPIWGGYS (SEQ ID NO: 20); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.C6 antibody described herein. As shown below, the 1E11.C6 antibody includes a heavy chain variable region (SEQ ID NO: 110) encoded by the nucleic acid sequence shown in SEQ ID NO: 109, and a light chain variable region (SEQ ID NO: 8) encoded by the nucleic acid sequence shown in SEQ ID NO: 80.

### 1E11.C6 VH Nucleic Acid Sequence

(SEQ ID NO: 109)

CAGGTGCAGCTTCAGGAGTCCGGCCCAGGACTGGTGAAGCCTTCGGACAC
CCTGTCCCTCACCTGCGCTGTCTCTGGTTACCCCATCGGCGGCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C6 VH Amino Acid Sequence

(SEQ ID NO: 110)

QVQLQESGPGLVKPSDTLSLTCAVSGYPIGGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C6 VL Nucleic Acid Sequence

(SEQ ID NO: 80)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGTCACAGTTTTCCGCTCACTTTCGGCGGA
GGGACCAAGGTGGAGATCAAA

### 1E11.C6 VL Amino Acid Sequence

(SEQ ID NO: 8)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C6 antibody have the following sequences: GYPIGGGYS (SEQ ID NO: 21); IHYSGYT (SEQ ID NO: 22); and ARKDSGNYFPY (SEQ ID NO: 24). The light chain CDRs of the 1E11.C1 antibody have the following sequences: QSISDH (SEQ ID NO: 34); YAS (SEQ ID NO: 35); and QQGHSFPLT (SEQ ID NO: 6).

An exemplary TLR4 monoclonal antibody is the 1E11.E1 antibody described herein. As shown below, the 1E11.E1 antibody includes a heavy chain variable region (SEQ ID NO: 78) encoded by the nucleic acid sequence shown in SEQ ID NO: 77, and a light chain variable region (SEQ ID NO: 112) encoded by the nucleic acid sequence shown in SEQ ID NO: 111.

### 1E11.E1 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.E1 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.E1 VL Nucleic Acid Sequence

(SEQ ID NO: 111)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGAACGACTTCCCGGTGACTTTCGGCGGA
GGGACCAAGGTGGAGATCAAA

### 1E11.E1 VL Amino Acid Sequence

(SEQ ID NO: 112)

EIVLTQSPDFQSVTPKEKVTITCRASQSISDHLHWYQQKPDQSPKLLIKY
ASHAISGVPSRFSGSGSGTDFTLTINSLEAEDAATYYCQQGNDFPVTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E1 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGNDFPVT (SEQ ID NO: 37).

An exemplary TLR4 monoclonal antibody is the 1E11.E2 antibody described herein. As shown below, the 1E11.E2 antibody includes a heavy chain variable region (SEQ ID NO: 78) encoded by the nucleic acid sequence shown in SEQ ID NO: 79, and a light chain variable region (SEQ ID NO: 114) encoded by the nucleic acid sequence shown in SEQ ID NO: 113.

### 1E11.E2 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.E2 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.E2 VL Nucleic Acid Sequence

(SEQ ID NO: 113)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGTACGACGAGCCGTTCACTTTCGGCGGA
GGGACCAAGGTGGAGATCAAA

### 1E11.E2 VL Amino Acid Sequence

(SEQ ID NO: 114)

EIVLTQSPDFQSVTPKEKVTITCRASQSISDHLHWYQQKPDQSPKLLIKY
ASHAISGVPSRFSGSGSGTDFTLTINSLEAEDAATYYCQQGYDEPFTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E2 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYDEPFT (SEQ ID NO: 38).

An exemplary TLR4 monoclonal antibody is the 1E11.E3 antibody described herein. As shown below, the 1E11.E3 antibody includes a heavy chain variable region (SEQ ID NO: 78) encoded by the nucleic acid sequence shown in SEQ ID NO: 79, and a light chain variable region (SEQ ID NO: 116) encoded by the nucleic acid sequence shown in SEQ ID NO: 115.

### 1E11.E3 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.E3 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.E3 VL Nucleic Acid Sequence

(SEQ ID NO: 115)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTTCCCGTTGACTTTCGGCGGA
GGGACCAAGGTGGAGATCAAA

### 1E11.E3 VL Amino Acid Sequence

(SEQ ID NO: 116)

EIVLTQSPDFQSVTPKEKVTITCRASQSISDHLHWYQQKPDQSPKLLIKY
ASHAISGVPSRFSGSGSGTDFTLTINSLEAEDAATYYCQQGYDFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E3 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYDFPLT (SEQ ID NO: 39).

An exemplary TLR4 monoclonal antibody is the 1E11.E4 antibody described herein. As shown below, the 1E11.E4 antibody includes a heavy chain variable region (SEQ ID NO: 79) encoded by the nucleic acid sequence shown in SEQ ID NO: 79, and a light chain variable region (SEQ ID NO: 118) encoded by the nucleic acid sequence shown in SEQ ID NO: 117.

### 1E11.E4 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.E4 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

1E11.E4 VL nucleic acid sequence

(SEQ ID NO: 117)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTACCCGCTCACTTTCGGCGGA
GGGACCAAGGTGGAGATCAAA

### 1E11.E4 VL Amino Acid Sequence

(SEQ ID NO: 118)

EIVLTQSPDFQSVTPKEKVTITCRASQSISDHLHWYQQKPDQSPKLLIKY
ASHAISGVPSRFSGSGSGTDFTLTINSLEAEDAATYYCQQGYDYPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E4 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYDYPLT (SEQ ID NO: 40).

An exemplary TLR4 monoclonal antibody is the 1E11.E5 antibody described herein. As shown below, the 1E11.E5 antibody includes a heavy chain variable region (SEQ ID NO: 78) encoded by the nucleic acid sequence shown in SEQ ID NO: 79, and a light chain variable region (SEQ ID NO: 120) encoded by the nucleic acid sequence shown in SEQ ID NO: 119.

### 1E11.E5 VH Nucleic Acid Sequence

(SEQ ID NO: 79)

CAGGTGCAGCTTCAGGAGTCCGGCCCAGGACTGGTGAAGCCTTCGGACAC
CCTGTCCCTCACCTGCGCTGTCTCTGGTTACTCCATCACCGGTGGTTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.E5 VH Amino Acid Sequence

(SEQ ID NO: 78)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.E5 VL Nucleic Acid Sequence

(SEQ ID NO: 119)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGAGTTCCCGTTGACTTTCGGCGGA
GGGACCAAGGTGGAGATCAAA

### 1E11.E5 VL Amino Acid Sequence

(SEQ ID NO: 120)

EIVLTQSPDFQSVTPKEKVTITCRASQSISDHLHWYQQKPDQSPKLLIKY
ASHAISGVPSRFSGSGSGTDFTLTINSLEAEDAATYYCQQGYEFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.E5 antibody have the following sequences: GYSITGGYS (SEQ ID NO: 15); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYEFPLT (SEQ ID NO: 41).

An exemplary TLR4 monoclonal antibody is the 1E11.C2E1 antibody described herein. As shown below, the 1E11.C2E1 antibody includes a heavy chain variable region (SEQ ID NO: 102) encoded by the nucleic acid sequence shown in SEQ ID NO: 101, and a light chain variable region (SEQ ID NO: 122) encoded by the nucleic acid sequence shown in SEQ ID NO: 121.

### 1E11.C2E1 VH Nucleic Acid Sequence

(SEQ ID NO: 101)

CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C2E1 VH Amino Acid Sequence

(SEQ ID NO: 102)

QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C2E1 VL Nucleic Acid Sequence

(SEQ ID NO: 121)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGGAACGACTTCCCGGTGACTTTCGGCGGA
GGGACCAAGGTGGAGATCAAA

### 1E11.C2E1 VL Amino Acid Sequence

(SEQ ID NO: 122)

EIVLTQSPDFQSVTPKEKVTITCRASQSISDHLHWYQQKPDQSPKLLIKY
ASHAISGVPSRFSGSGSGTDFTLTINSLEAEDAATYYCQQGNDFPVTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E1 antibody have the following sequences: GYPIRFGYS (SEQ ID NO: 17); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGNDFPVT (SEQ ID NO: 37).

An exemplary TLR4 monoclonal antibody is the 1E11.C2E3 antibody described herein. As shown below, the 1E11.C2E3 antibody includes a heavy chain variable region (SEQ ID NO: 102) encoded by the nucleic acid sequence shown in SEQ ID NO: 101, and a light chain variable region (SEQ ID NO: 124) encoded by the nucleic acid sequence shown in SEQ ID NO: 123.

### 1E11.C2E3 VH Nucleic Acid Sequence

(SEQ ID NO: 101)

CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C2E3 VH Amino Acid Sequence

(SEQ ID NO: 102)

QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C2E3 VL Nucleic Acid Sequence

(SEQ ID NO: 123)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTTCCCGTTGACTTTCGGCGGA
GGGACCAAGGTGGAGATCAAA

### 1E11.C2E3 VL Amino Acid Sequence

(SEQ ID NO: 124)

EIVLTQSPDFQSVTPKEKVTITCRASQSISDHLHWYQQKPDQSPKLLIKY
ASHAISGVPSRFSGSGSGTDFTLTINSLEAEDAATYYCQQGYDFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1P.37 (2000) LIGM:230). The heavy chain CDRs of the 1E11.C2E3 antibody have the following sequences: GYPIRFGYS (SEQ ID NO: 17); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYDFPLT (SEQ ID NO: 39).

An exemplary TLR4 monoclonal antibody is the 1E11.C2E4 antibody described herein. As shown below, the 1E11.C2E4 antibody includes a heavy chain variable region (SEQ ID NO: 102) encoded by the nucleic acid sequence shown in SEQ ID NO: 101, and a light chain variable region (SEQ ID NO: 126) encoded by the nucleic acid sequence shown in SEQ ID NO: 125.

### 1E11.C2E4 VH Nucleic Acid Sequence

(SEQ ID NO: 101)

CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C2E4 VH Amino Acid Sequence

(SEQ ID NO: 102)

QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C2E4 VL Nucleic Acid Sequence

(SEQ ID NO: 125)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGACTACCCGCTCACTTTCGGCGGA
GGGACCAAGGTGGAGATCAAA

### 1E11.C2E4 VL Amino Acid Sequence

(SEQ ID NO: 126)

EIVLTQSPDFQSVTPKEKVTITCRASQSISDHLHWYQQKPDQSPKLLIKY
ASHAISGVPSRFSGSGSGTDFTLTINSLEAEDAATYYCQQGYDYPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.1 P.37 (2000) LIGM:230). The heavy chain CDRs of the 1E11.C2E4 antibody have the following sequences: GYPIRFGYS (SEQ ID NO: 17); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYDYPLT (SEQ ID NO: 40).

An exemplary TLR4 monoclonal antibody is the 1E11.C2E5 antibody described herein. As shown below, the 1E11.C2E5 antibody includes a heavy chain variable region (SEQ ID NO: 102) encoded by the nucleic acid sequence shown in SEQ ID NO: 101, and a light chain variable region (SEQ ID NO: 128) encoded by the nucleic acid sequence shown in SEQ ID NO: 127.

### 1E11.C2E5 VH Nucleic Acid Sequence

(SEQ ID NO: 101)

CAGGTGCAGCTTCAGGAGTCCGGCCCAGGACTGGTGAAGCCTTCGGACAC
CCTGTCCCTCACCTGCGCTGTCTCTGGTTACCCGATCCGGTTCGGCTATA
GCTGGCACTGGATACGGCAGCCCCCAGGGAAGGGACTGGAGTGGATGGGG
TATATCCACTACAGTGGTTACACTGACTTCAACCCCTCCCTCAAGACTCG
AATCACCATATCACGTGACACGTCCAAGAACCAGTTCTCCCTGAAGCTGA
GCTCTGTGACCGCTGTGGACACTGCAGTGTATTACTGTGCGAGAAAAGAT
TCGGGCAACTACTTCCCTTACTGGGGCCAAGGGACTCTGGTCACTGTCTC
TTCC

### 1E11.C2E5 VH Amino Acid Sequence

(SEQ ID NO: 102)

QVQLQESGPGLVKPSDTLSLTCAVSGYPIRFGYSWHWIRQPPGKGLEWMG
YIHYSGYTDFNPSLKTRITISRDTSKNQFSLKLSSVTAVDTAVYYCARKD
SGNYFPYWGQGTLVTVSS

### 1E11.C2E5 VL Nucleic Acid Sequence

(SEQ ID NO: 127)

GAAATTGTGTTGACGCAGTCTCCAGACTTTCAGTCTGTGACTCCAAAGGA
AAAAGTCACCATCACCTGCAGGGCCAGTCAGAGTATCAGCGACCACTTAC
ACTGGTACCAACAGAAACCTGATCAGTCTCCCAAGCTCCTCATCAAATAT
GCTTCCCATGCCATTTCTGGGGTCCCATCGAGGTTCAGTGGCAGTGGGTC
TGGGACAGACTTCACTCTCACCATCAATAGCCTAGAGGCTGAAGATGCTG
CAACGTATTACTGTCAGCAGGGCTACGAGTTCCCGTTGACTTTCGGCGGA
GGGACCAAGGTGGAGATCAAA

### 1E11.C2E5 VL Amino Acid Sequence

(SEQ ID NO: 128)

EIVLTQSPDFQSVTPKEKVTITCRASQSISDHLHWYQQKPDQSPKLLIKY
ASHAISGVPSRFSGSGSGTDFTLTINSLEAEDAATYYCQQGYEFPLTFGG
GTKVEIK

The amino acids encompassing the complementarity determining regions (CDR) are as defined by M. P. Lefranc (See Lefranc, M.-P., Current Protocols in Immunology, J. Wiley and Sons, New York supplement 40, A1.P.1-A.IP.37 (2000) LIGM:230). The heavy chain CDRs of the 1E11.C2E5 antibody have the following sequences: GYPIRFGYS (SEQ ID NO: 17); IHYSGYT (SEQ ID NO: 22); and ARKDSGNYFPY (SEQ ID NO: 24). The light chain CDRs of the 1E11 antibody have the following sequences: QSISDH (SEQ ID NO: 34); YAS (SEQ ID NO: 35); and QQGYEFPLT (SEQ ID NO: 41).

In some embodiments, the TLR4 antibodies are formatted in an IgG isotype. In some embodiments, the TLR4 antibodies are formatted in an IgG1 isotype.

An exemplary IgG1-formatted antibody is the IgG1-formatted 1E11 antibody comprising the heavy chain sequence of SEQ ID NO: 130 and the light chain sequence of SEQ ID NO: 132, as shown below:

### 1E11 Heavy Chain Amino Acid Sequence

(SEQ ID NO: 130)

QVQLQESGPGLVKPSDTLSLTCAVSGYSITGGYSWHWIRQPPGKGLEWMG
YIHYSGYTDFNPSLKTRITISRDTSKNQFSLKLSSVTAVDTAVYYCARKD
SGNYFPYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG

### 1E11 Light Chain Amino Acid Sequence

(SEQ ID NO: 132)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### 1E11 Light Chain Nucleic Acid Sequence

(SEQ ID NO: 131)

ATGAGTGTGCCCACTCAGGTCCTGGGGTTGCTGCTGCTGTGGCTTACAGA
TGCCAGATGTGAAATTGTGTTGACGCAGTCTCCAGACTTTCAGTCTGTGA
CTCCAAAGGAAAAAGTCACCATCACCTGCAGGGCCAGTCAGAGTATCAGC
GACCACTTACACTGGTACCAACAGAAACCTGATCAGTCTCCCAAGCTCCT
CATCAAATATGCTTCCCATGCCATTTCTGGGGTCCCATCGAGGTTCAGTG
GCAGTGGGTCTGGGACAGACTTCACTCTCACCATCAATAGCCTAGAGGCT
GAAGATGCTGCAACGTATTACTGTCAGCAGGGTCACAGTTTTCCGCTCAC
TTTCGGCGGAGGGACCAAGGTGGAGATCAAACGTACGGTGGCTGCACCAT
CTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCA
CAGAGCAGGACAGCAAGGACAGCACCTACAGCCTCAGCAGCACCCTGACG
CTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGT
GTTAA

### 1E11 Heavy Chain Nucleic Acid Sequence

(SEQ ID NO: 129)

ATGGAATGGAGCTGGGTCTTTCTCTTCTTCCTGTCAGTAACTACAGGTGT
CCACCAGGTGCAGCTTCAGGAGTCCGGCCCAGGACTGGTGAAGCCTTCGG
ACACCCTGTCCCTCACCTGCGCTGTCTCTGGTTACTCCATCACCGGTGGT
TATAGCTGGCACTGGATACGGCAGCCCCCAGGGAAGGGACTGGAGTGGAT
GGGGTATATCCACTACAGTGGTTACACTGACTTCAACCCCTCCCTCAAGA
CTCGAATCACCATATCACGTGACACGTCCAAGAACCAGTTCTCCCTGAAG
CTGAGCTCTGTGACCGCTGTGGACACTGCAGTGTATTACTGTGCGAGAAA
AGATCCGTCCGACGCCTTTCCTTACTGGGGCCAAGGGACTCTGGTCACTG
TCTCTTCCGCCTCCACCAAGGGCCCATCGGTCTTCCCCCTGGCACCCTCC
TCCAAGAGCACCTCTGGGGGCACAGCGGCCCTGGGCTGCCTGGTCAAGGA
CTACTTCCCCGAACCGGTGACAGTCTCGTGGAACTCAGGAGCCCTGACCA
GCGGCGTGCACACCTTCCCGGCTGTCCTACAGTCCTCAGGACTCTACTCC
CTCAGCAGCGTGGTGACTGTGCCCTCCAGCAGCTTGGGCACCCAGACCTA
CATCTGCAACGTGAATCACAAGCCCAGCAACACCAAGGTGGACAAGAGAG
TTGAGCCCAAATCTTGTGACAAAACTCACACATGCCCACCGTGCCCAGCA
CCTGAACTCCTGGGGGGACCGTCAGTCTTCCTCTTCCCCCCAAAACCCAA
GGACACCCTCATGATCTCCCGGACCCCTGAGGTCACATGCGTGGTGGTGG
ACGTGAGCCACGAAGACCCTGAGGTCAAGTTCAACTGGTACGTGGACGGC
GTGGAGGTGCATAATGCCAAGACAAAGCCGCGGGAGGAGCAGTACAACAG
CACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGT
GTATACCCTGCCCCCATCTCGGGAGGAGATGACCAAGAACCAGGTCAGCC
TGACTTGCCTGGTCAAAGGCTTCTATCCCAGCGACATCGCCGTGGAGTGG
GAGAGCAACGGGCAGCCGGAGAACAACTACAAGACCACGCCTCCCGTGCT
GGACTCCGACGGCTCCTTCTTCCTCTATAGCAAGCTCACCGTGGACAAGT
CCAGGTGGCAGCAGGGGAACGTCTTCTCATGCTCCGTGATGCATGAGGCT
CTGCACAACCACTACACGCAGAAGAGCCTCTCCCTGTCTCCGGGTTAA

An exemplary IgG1-formatted antibody is the IgG1-formatted 1E11.C11 antibody comprising the heavy chain sequence of SEQ ID NO: 134 and the light chain sequence of SEQ ID NO: 136, as shown below:

### 1E11.C1 Light Chain Amino Acid Sequence

(SEQ ID NO: 136)

EIVLTQSPDFQSVTPKEKVTITCRASQSISDHLHWYQQKPDQSPKLLIKY
ASHAISGVPSRFSGSGSGTDFTLTINSLEAEDAATYYCQQGHSFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

### 1E11.C1 Heavy Chain Amino Acid Sequence

(SEQ ID NO: 134)

QVQLQESGPGLVKPSDTLSLTCAVSGFPIRYGYSWHWIRQPPGKGLEWMG
YIHYSGYTDFNPLKTRITISRDTSKNQFSLKLSSVTAVDTAVYYCARKDS
GNYFPYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG

### 1E11.C1 Light Chain Nucleic Acid Sequence

(SEQ ID NO: 135)

ATGAGTGTGCCCACTCAGGTCCTGGGGTTGCTGCTGCTGTGGCTTACAGA
TGCCAGATGTGAAATTGTGTTGACGCAGTCTCCAGACTTTCAGTCTGTGA
CTCCAAAGGAAAAAGTCACCATCACCTGCAGGGCCAGTCAGAGTATCAGC
GACCACTTACACTGGTACCAACAGAAACCTGATCAGTCTCCCAAGCTCCT
CATCAAATATGCTTCCCATGCCATTTCTGGGGTCCCATCGAGGTTCAGTG
GCAGTGGGTCTGGGACAGACTTCACTCTCACCATCAATAGCCTAGAGGCT
GAAGATGCTGCAACGTATTACTGTCAGCAGGGTCACAGTTTTCCGCTCAC
TTTCGGCGGAGGGACCAAGGTGGAGATCAAACGTACGGTGGCTGCACCAT
CTGTCTTCATCTTCCCGCCATCTGATGAGCAGTTGAAATCTGGAACTGCC
TCTGTTGTGTGCCTGCTGAATAACTTCTATCCCAGAGAGGCCAAAGTACA
GTGGAAGGTGGATAACGCCCTCCAATCGGGTAACTCCCAGGAGAGTGTCA
CAGAGCAGGACAGCAAGGACAGCACCTACAGCCTCAGCAGCACCCTGACG
CTGAGCAAAGCAGACTACGAGAAACACAAAGTCTACGCCTGCGAAGTCAC
CCATCAGGGCCTGAGCTCGCCCGTCACAAAGAGCTTCAACAGGGGAGAGT
GTTAA

### 1E11.C1 Heavy Chain Nucleic Acid Sequence

(SEQ ID NO: 133)

ATGGAATGGAGCTGGGTCTTTCTCTTCTTCCTGTCAGTAACTACAGGTGT
CCACCAGGTGCAGCTTCAGGAGTCCGGCCCAGGACTGGTGAAGCCTTCGG
ACACCCTGTCCCTCACCTGCGCTGTCTCTGGTTTCCCGATCCGCTACGGG
TATAGCTGGCACTGGATACGGCAGCCCCCAGGGAAGGGACTGGAGTGGAT
GGGGTATATCCACTACAGTGGTTACACTGACTTCAACCCCTCCCTCAAGA
CTCGAATCACCATATCACGTGACACGTCCAAGAACCAGTTCTCCCTGAAG
CTGAGCTCTGTGACCGCTGTGGACACTGCAGTGTATTACTGTGCGAGAAA
AGATTCGGGCAACTACTTCCCTTACTGGGGCCAAGGGACTCTGGTCACTG
TCTCTTCCGCCTCCACCAAGGGCCCATCGGTCTTCCCCCTGGCACCCTCC
TCCAAGAGCACCTCTGGGGGCACAGCGGCCCTGGGCTGCCTGGTCAAGGA
CTACTTCCCCGAACCGGTGACAGTCTCGTGGAACTCAGGAGCCCTGACCA
GCGGCGTGCACACCTTCCCGGCTGTCCTACAGTCCTCAGGACTCTACTCC
CTCAGCAGCGTGGTGACTGTGCCCTCCAGCAGCTTGGGCACCCAGACCTA
CATCTGCAACGTGAATCACAAGCCCAGCAACACCAAGGTGGACAAGAGAG
TTGAGCCCAAATCTTGTGACAAAACTCACACATGCCCACCGTGCCCAGCA
CCTGAACTCCTGGGGGGACCGTCAGTCTTCCTCTTCCCCCCAAAACCCAA
GGACACCCTCATGATCTCCCGGACCCCTGAGGTCACATGCGTGGTGGTGG
ACGTGAGCCACGAAGACCCTGAGGTCAAGTTCAACTGGTACGTGGACGGC
GTGGAGGTGCATAATGCCAAGACAAAGCCGCGGGAGGAGCAGTACAACAG
CACGTACCGTGTGGTCAGCGTCCTCACCGTCCTGCACCAGGACTGGCTGA
ATGGCAAGGAGTACAAGTGCAAGGTCTCCAACAAAGCCCTCCCAGCCCCC
ATCGAGAAAACCATCTCCAAAGCCAAAGGGCAGCCCCGAGAACCACAGGT
GTATACCCTGCCCCCATCTCGGGAGGAGATGACCAAGAACCAGGTCAGCC
TGACTTGCCTGGTCAAAGGCTTCTATCCCAGCGACATCGCCGTGGAGTGG
GAGAGCAACGGGCAGCCGGAGAACAACTACAAGACCACGCCTCCCGTGCT
GGACTCCGACGGCTCCTTCTTCCTCTATAGCAAGCTCACCGTGGACAAGT
CCAGGTGGCAGCAGGGGAACGTCTTCTCATGCTCCGTGATGCATGAGGCT
CTGCACAACCACTACACGCAGAAGAGCCTCTCCCTGTCTCCGGGTTAA

In some embodiments, TLR4 antibodies of the invention specifically bind human and/or cynomolgus TLR4/MD-2 complex, wherein the antibody binds to an epitope that includes one or more amino acid residues on human and/or cynomolgus TLR4 between residues 325 and 374 of SEQ ID NO: 76 (human) and SEQ ID NO: 77 (cynomolgus). Alternatively, the monoclonal antibody is an antibody that binds to the same epitope as 1A1, 1A6, 1B12, 1C7, 1C10, 1C12, 1D10, 1E11, 1E11 N103D, 1G12, 1E11.C1, 1E11.C2, 1E11.C3, 1E11.C4, 1E11.C5, 1E11.C6, 1E11.E1, 1E11.E2, 1E11.E3, 1E11.E4, 1E11.E5, 1E11.C2E1, 1E11.C2E3, 1E11.C2E4 and 1E11.C2E5.

The anti-TLR4 antibodies of the invention include an altered antibody in which at least the amino acid residue at EU position 325 and at least the amino acid residue at EU position 328 in the CH2 domain of the Fc portion of the antibody has been modified. For example, at least the amino acid residue at EU position 325 has been substituted with serine, and at least the amino acid residue at EU position 328 has been substituted with phenylalanine.

These anti-TLR4 antibodies with a modified Fc portion elicit modified effector functions e.g., a modified Fc receptor activity, as compared to an unaltered antibody. For example, the human Fc receptor is CD32A. In some embodiments, these anti-TLR4 antibodies elicit a prevention of proinflammatory mediators release following ligation to CD32A as compared to an unaltered antibody. Thus, these anti-TLR4 antibodies elicit a modified Fc receptor activity, such as the prevention of proinflammatory mediators release while retaining the ability to bind a target antigen. In some embodiments, these anti-TLR4 antibodies are neutralizing antibodies, wherein the anti-TLR4 antibody elicits a modified Fc receptor activity, while retaining the ability to neutralize one or more biological activities of a target antigen.

For example, anti-TLR4 antibodies of the invention include monoclonal antibodies that bind the human TLR4/MD-2 receptor complex. This receptor complex is activated by lipopolysaccharide (LPS), the major component of the outer membrane of gram-negative bacteria. The anti-TLR4 antibodies of the invention inhibit receptor activation and subsequent intracellular signaling via LPS. Thus, the anti-TLR4 antibodies neutralize the activation of the TLR4/MD-2 receptor complex. In particular, the invention provides anti-TLR4 antibodies that recognize the TLR4/MD-2 receptor complex expressed on the cell surface. These anti-TLR4 antibodies block LPS-induced and other TLR4 ligand-induced pro-inflammatory cytokine (e.g., IL-6, IL-8, TNFα) production. In addition, some anti-TLR4 antibodies of the invention also recognize TLR4 when not complexed with MD-2. The altered antibody is, e.g., a humanized antibody.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of; cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of; analytical chemistry, synthetic ‘organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Use of Anti-TLR4 Antibodies

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed., Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery−some emerging concepts.” J Pharm Sci. 89(8):967-78 (2000), Powell et al. “Compendium of excipients for parenteral formulations” PDA J Pharm Sci Technol. 52:238-311(1998) and the citations therein for additional information related to formulations, excipients and carriers well known to pharmaceutical chemists.

Therapeutic formulations of the invention, which include an anti-TLR4 antibody of the invention, are used to treat or alleviate a symptom associated with an immune-related disorder. The present invention also provides methods of treating or alleviating a symptom associated with an immune-related disorder. A therapeutic regimen is carried out by identifying a subject, e.g., a human patient suffering from (or at risk of developing) an immune-related disorder, using standard methods. For example, anti-TLR4 antibodies of the invention are useful therapeutic tools in the treatment of autoimmune diseases and/or inflammatory disorders. In certain embodiments, the use of anti-TLR4 antibodies that modulate, e.g., inhibit, neutralize, or interfere with, TLR signaling is contemplated for treating autoimmune diseases and/or inflammatory disorders.

Autoimmune diseases include, for example, Acquired Immunodeficiency Syndrome (AIDS, which is a viral disease with an autoimmune component), alopecia areata, ankylosing spondylitis, antiphospholipid syndrome, autoimmune Addison's disease, autoimmune hemolytic anemia, autoimmune hepatitis, autoimmune inner ear disease (AIED), autoimmune lymphoproliferative syndrome (ALPS), autoimmune thrombocytopenic purpura (ATP), Behcet's disease, cardiomyopathy, celiac sprue-dermatitis hepetiformis; chronic fatigue immune dysfunction syndrome (CFIDS), chronic inflammatory demyelinating polyneuropathy (CIPD), cicatricial pemphigold, cold agglutinin disease, crest syndrome, Crohn's disease, Degos' disease, dermatomyositis-juvenile, discoid lupus, essential mixed cryoglobulinemia, fibromyalgia-fibromyositis, Graves' disease, Guillain-Barre syndrome, Hashimoto's thyroiditis, idiopathic pulmonary fibrosis, idiopathic thrombocytopenia purpura (ITP), IgA nephropathy, insulin-dependent diabetes mellitus, juvenile chronic arthritis (Still's disease), juvenile rheumatoid arthritis, Meniere's disease, mixed connective tissue disease, multiple sclerosis, myasthenia gravis, pernacious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomena, Reiter's syndrome, rheumatic fever, rheumatoid arthritis, sarcoidosis, scleroderma (progressive systemic sclerosis (PSS), also known as systemic sclerosis (SS)), Sjogren's syndrome, stiff-man syndrome, systemic lupus erythematosus, Takayasu arteritis, temporal arteritis/giant cell arteritis, ulcerative colitis, uveitis, vitiligo and Wegener's granulomatosis.

Inflammatory disorders include, for example, chronic and acute inflammatory disorders. Examples of inflammatory disorders include Alzheimer's disease, asthma, atopic allergy, allergy, atherosclerosis, bronchial asthma, eczema, glomerulonephritis, graft vs. host disease, hemolytic anemias, osteoarthritis, sepsis, stroke, transplantation of tissue and organs, vasculitis, diabetic retinopathy and ventilator induced lung injury.

For example, anti-TLR4 antibodies are useful in the treatment of acute inflammation and sepsis induced by microbial products (e.g., LPS) and exacerbations arising from this acute inflammation, such as, for example, chronic obstructive pulmonary disease and asthma (see O'Neill, Curr. Opin. Pharmacol. 3: 396-403 (2003), hereby incorporated by reference in its entirety). Such antibodies are also useful in treating neurodegenerative autoimmune diseases. (Lehnardt et al., Proc. Natl. Acad. Sci. USA 100: 8514-8519(2003), hereby incorporated by reference in its entirety).

In addition, the antibodies of the invention are also useful as therapeutic reagents in the treatment of diseases, such as, for example, osteoarthritis, which are caused by stress, for example, cellular stress, which, in turn, induces endogenous soluble “stress” factors that trigger TLR4. Endogenous soluble stress factor include e.g., Hsp60 (see Ohashi et al., J. Immunol. 164: 558 561 (2000)) and fibronectin (see Okamura et al., J. Biol. Chem. 276:10229 10233 (2001) and heparin sulphate, hyaluronan, gp96, [3 Defensin-2 or surfactant protein A (see e.g., Johnson et al., Crit. Rev. Immunol., 23(1-2):15-44 (2003), each of which is hereby incorporated by reference in its entirety). The antibodies of the invention are also useful in the treatment of a variety of disorders associated with stress, such as for example, cellular stress that is associated with subjects and patients placed on respirators, ventilators and other respiratory assist devices. For example, the antibodies of the invention are useful in the treatment of ventilator-induced lung injury (“VILI”), also referred to as ventilation-associated lung injury (“VALI”).

Other disease areas in which inhibiting TLR4 function could be beneficial include, for example, chronic inflammation (e.g., chronic inflammation associated with allergic conditions and asthma), autoimmune diseases (e.g., inflammatory bowel disorder) and atherosclerosis (see O'Neill, Curr. Opin. Pharmacol. 3: 396-403 (2003), hereby incorporated by reference in its entirety).

Symptoms associated with these immune-related disorders include, for example, inflammation, fever, general malaise, fever, pain, often localized to the inflamed area, rapid pulse rate, joint pain or aches (arthralgia), rapid breathing or other abnormal breathing patterns, chills, confusion, disorientation, agitation, dizziness, cough, dyspnea, pulmonary infections, cardiac failure, respiratory failure, edema, weight gain, mucopurulent relapses, cachexia, wheezing, headache, and abdominal symptoms such as, for example, abdominal pain, diarrhea or constipation.

Efficaciousness of treatment is determined in association with any known method for diagnosing or treating the particular immune-related disorder. Alleviation of one or more symptoms of the immune-related disorder indicates that the antibody confers a clinical benefit.

Antibodies of the invention, including polyclonal, monoclonal, humanized and fully human antibodies, may be used as therapeutic agents. Such agents will generally be employed to treat or prevent a disease or pathology associated with aberrant expression or activation of a given target in a subject. An antibody preparation, preferably one having high specificity and high affinity for its target antigen, is administered to the subject and will generally have an effect due to its binding with the target. Administration of the antibody may abrogate or inhibit or interfere with the signaling function of the target. Administration of the antibody may abrogate or inhibit or interfere with the binding of the target with an endogenous ligand to which it naturally binds. For example, the antibody binds to the target and neutralizes TLR4 ligand-induced proinflammatory cytokine production.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies or a fragment thereof of the invention can be administered for the treatment of a variety of diseases and disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

The pharmaceutical compositions can be included in a container, pack, or dispenser together with instructions for administration.

The formulation can also contain more than one active compound, e.g., anti-TLR4 antagonist as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

In one embodiment, the active compound, e.g., an anti-TLR4 antagonist, is administered in combination therapy, i.e., combined with one or more additional agents that are useful for treating pathological conditions or disorders, such as various forms of cancer, autoimmune disorders and inflammatory diseases. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

For example, the combination therapy can include one or more neutralizing anti-TLR4 antibodies of the invention coformulated with, and/or coadministered with, one or more additional therapeutic agents, e.g., one or more cytokine and growth factor inhibitors, immunosuppressants, anti-inflammatory agents, metabolic inhibitors, enzyme inhibitors, and/or cytotoxic or cytostatic agents, as described in more detail below. Such combination therapies may advantageously utilize lower dosages of the administered therapeutic agents, thus avoiding possible toxicities or complications associated with the various monotherapies.

Preferred therapeutic agents used in combination with a neutralizing anti-TLR4 antibody of the invention are those agents that interfere at different stages in an inflammatory response. In one embodiment, one or more neutralizing anti-TLR4 antibodies described herein may be coformulated with, and/or coadministered with, one or more additional agents such as other cytokine or growth factor antagonists (e.g., soluble receptors, peptide inhibitors, small molecules, ligand fusions); or antibodies or antigen binding fragments thereof that bind to other targets (e.g., antibodies that bind to other cytokines or growth factors, their receptors, or other cell surface molecules); and anti-inflammatory cytokines or agonists thereof

Where antibody fragments are used, the smallest inhibitory fragment that specifically binds to the binding domain of the target protein and/or the smallest inhibitory fragment that interferes with or otherwise antagonizes TLR4 signaling is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

Levels of TLR4 ligands and other related biomarkers are detecting using any of a variety of standard detection techniques. Detection agents can be used for detecting the presence of a given target (or a protein fragment thereof) in a sample. In some embodiments, the detection agent contains a detectable label. In some embodiments, the detection agent is an antibody (or fragment thereof) or a probe. In some embodiments, the agent or probe is labeled. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin.

The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. The bodily fluids can be fluids isolated from anywhere in the body of the subject, preferably a peripheral location, including but not limited to, for example, blood, plasma, serum, synovial fluid, urine, sputum, spinal fluid, cerebrospinal fluid, pleural fluid, fluid of the respiratory, intestinal, and genitourinary tracts, saliva, intra-organ system fluid, ascitic fluid, tumor cyst fluid, amniotic fluid and combinations thereof. The biological sample also includes experimentally separated fractions of all of the preceding fluids. Biological samples also include solutions or mixtures containing homogenized solid material, such as feces, tissues, and biopsy samples. The detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbant assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Pharmaceutical Compositions

The antibodies or soluble chimeric polypeptides of the invention (also referred to herein as “active compounds”), and derivatives, fragments, analogs and homologs thereof, can be incorporated into pharmaceutical compositions suitable for administration. Such compositions typically comprise the antibody or soluble chimeric polypeptide and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention will be further described in the following examples, which do not limit the scope of the invention described in the claims.

### EXAMPLES

### Example 1

### Cytokine Production in Rheumatoid Arthritis Synovial Fluid Samples Treated with Anti-TLR4 Antibodies

The studies presented herein were designed to evaluate the effect of treatment with an anti-TLR4 antibody of the disclosure, NI-0101, on cytokine production in rheumatoid arthritis synovial fluid (RASF) samples isolated from rheumatoid arthritis (RA) patients.

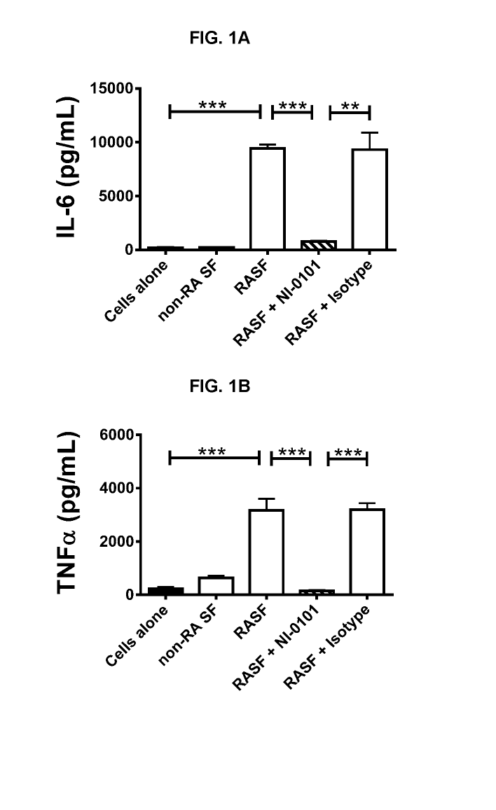

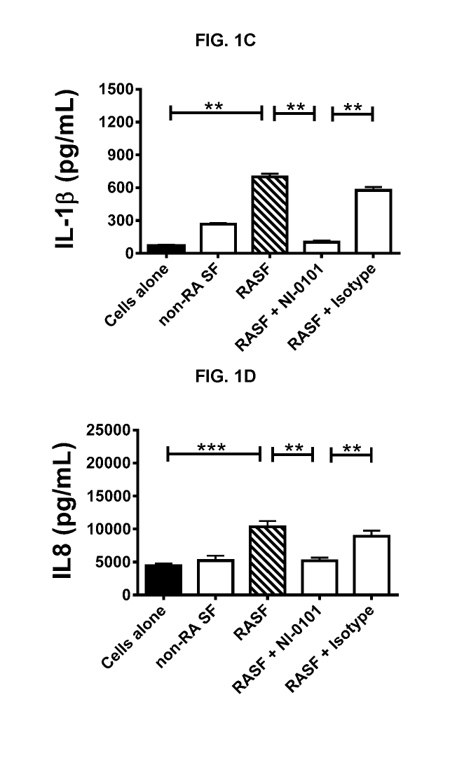

As shown in FIGS. 1A-1D, treatment with the anti-TLR4 antibody NI-0101 blocked IL-6 (FIG. 1A), TNFα (FIG. 1B), IL-1β (FIG. 1C) and IL-8 (FIG. 1D) production from pooled RASF-stimulated monocytes isolated from RA patients. TLR4 signaling was blocked with the anti-human TLR4 monoclonal antibody NI-0101.

### Example 2

### Identification of Responders and Non-Responders to Anti-TLR4 Antibody Treatment

The studies presented herein were designed to evaluate the capacity of RASF samples to stimulate cytokine production and respond to TLR4 blockade. RASF samples from patients (“Pat”) were classified as NI-0101 responders (“R”) if NI-0101 was able to block (partially or totally) RASF-induced IL6 production from RA monocytes. Others were classified as NI-0101 non-responders (NR). Of the 36 RASF samples tested, 18 were classified as NI-0101 responders (50%) and 18 as NI-0101 responders (50%).

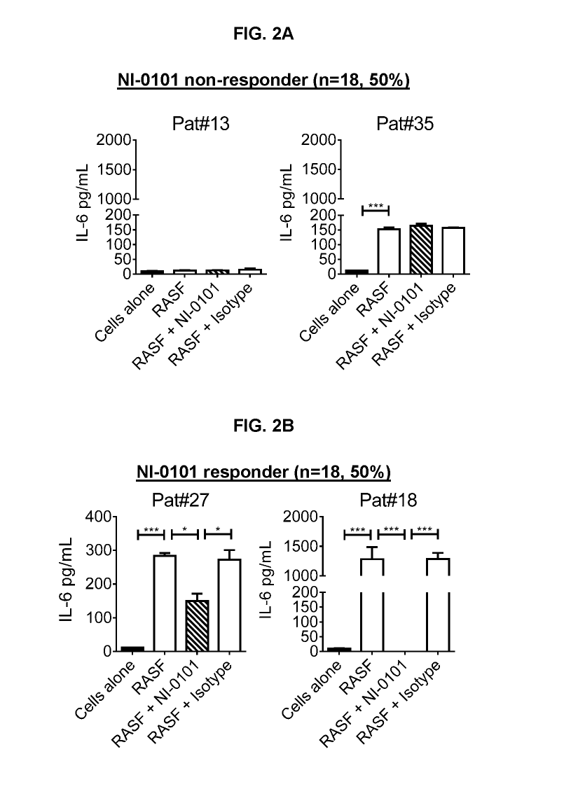

FIGS. 2A and 2B present representative examples of non-responder RASF and responder RASF patients. As shown in FIGS. 2A-2B, heterogeneity was seen among the synovial fluid samples.

### Example 3

### Expression Levels of ACPA and TLR4 Ligands in Synovial Fluid Samples

The studies presented herein were designed to evaluate the expression levels of ACPA and the TLR4 ligands HMGB1 and S100A8/A9 in the synovial fluid samples of non-rheumatoid arthritis patients and RA patients and their correlation with NI-0101 response.

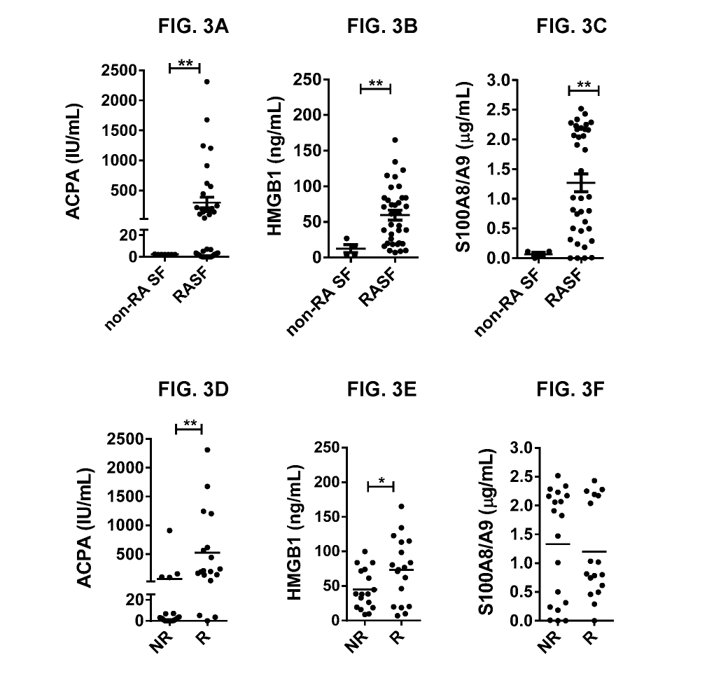

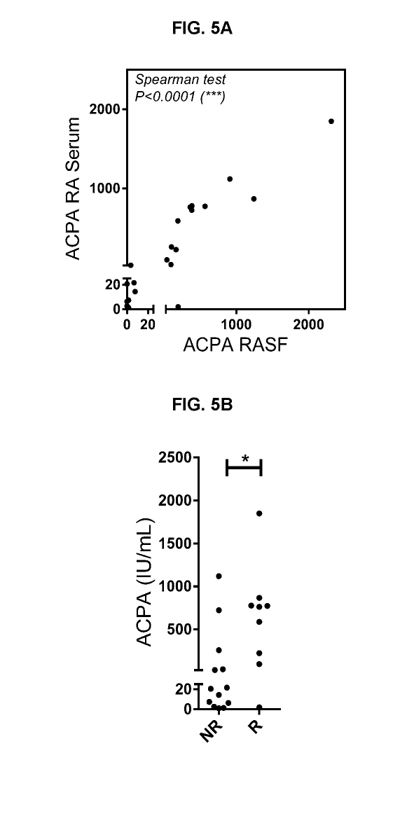

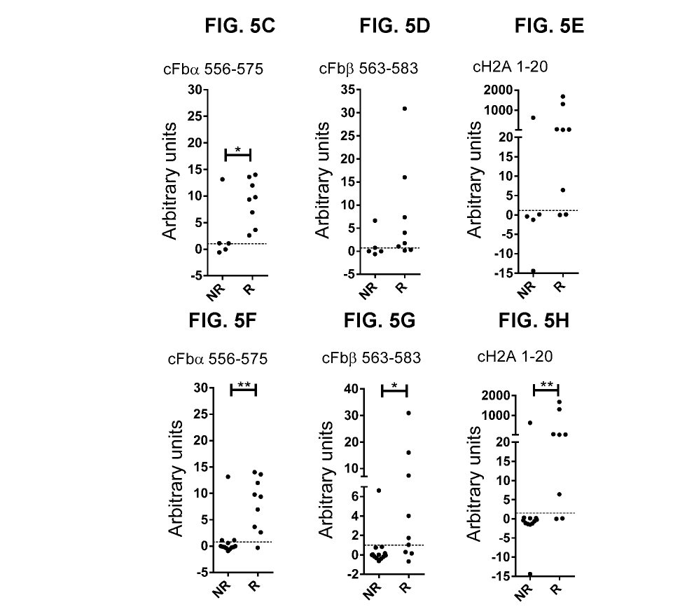

As shown in FIGS. 3A-3C, the TLR4 ligands anti-citrullinated protein antibody (ACPA), HMGB1, and 5100A8/A9 are present in elevated levels in RASF samples, but not in non-RA patient synovial fluid samples. As shown in FIGS. 3D-3F, the levels of ACPA, HMGB1, and S100A8/A9 were correlated with NI-0101 responder status. In particular, ACPA expression levels were found to be enriched in the NI-0101 responder group (FIG. 3D), and a difference in expression level of HMGB1 was detected in NI-0101 responders versus non-responders (FIG. 5E).

### Example 4

### Expression of Antibodies Against Citrullinated Peptides in Synovial Fluid Samples from RA Patients

The studies presented herein were designed to evaluate the ACPA expression profile in individuals as a predictor of NI-0101 response. To better characterize the correlation, the reactivity to the following citrullinated peptides derived from fibrinogen-α (cFbα 556-575), fibrinogen-β (cFbβ 563-583), and histone-2A (cH2A 1-20) was evaluated.

TABLE 1
Amino acids (AA) sequences of the citrullinated
peptides used to assess ACPA specificities. AA
names are given in their letters code.
Cit = citrulline

| Peptides | Protein | Amino Acid sequences |
|---|---|---|
| cFbα 556-575 | Fibrinogen | NTKESSSHHPGIAEFPS-Cit-GK |
| | | (SEQ ID NO: 1) |
| citFbβ 563-583 | Fibrinogen | HHPGIAEFPS-Cit- |
| | | GKSSSYSKQF |
| | | (SEQ ID NO: 2) |
| citH2A 1-20 | Histone 2A | MSG-Cit-GKQGGKA-Cit- |
| | | AKAKS-Cit-SS |
| | | (SEQ ID NO: 3) |

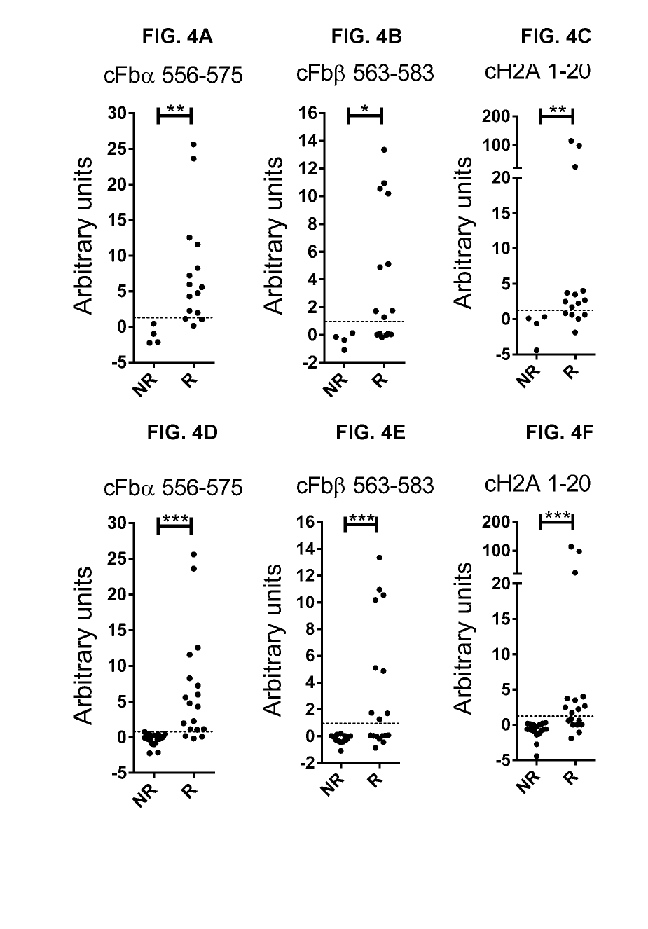

As shown in FIGS. 4A-4F, antibody reactivity against the citrullinated peptides was determined by ELISA in synovial fluids from RA patients and correlated with response to NI-0101. The level of reactivity to the citrullinated peptides in ACPA⁺ patients is shown in FIGS. 4A-4C, while FIGS. 4D-4F shown the level of activity in all patients, i.e., both ACPA⁺ and ACPA⁻ patients. Activity was enriched for responders as compared to non-responders.

### Example 5

### Expression of Antibodies Against Citrullinated Peptides in Serum Samples and Synovial Fluid Samples from RA Patients

The studies presented herein were designed to evaluate ACPA fine specificity in paired sera samples of RA patients and their correlation with RASF response to NI-0101.

The correlation between ACPA levels in paired RA sera and synovial fluids (n=22) was evaluated, and the results are shown in FIG. 5A. ACPA levels in paired RA sera classified according to RASF response to NI-0101 (NI-0101 non-responders (NR) or NI-0101 responders (R) was evaluated, and the results are shown in FIG. 5B. Antibody reactivity against the citrullinated peptides derived from fibrinogen-α (cFbα 556-575), fibrinogen-β (cFbβ 563-583) and histone-2A (cH2A 1-20) were determined by ELISA in paired sera from RA patients and correlated with response to NI-0101. The sensitivity and specificity of these markers are shown in FIGS. 5C-5H and below in Table 2. Sensitivity in this context is defined as the percentage of NI-0101 responders identified as positive in the assay, and specificity is defined as the percentage of NI-0101 non-responders identified as negative in the assay.

TABLE 2
The sensitivity and specificity of the antibody reactivity in
RA sera to individual citrullinated peptides and their combinations
to predict NI-0101 response (based on data from FIG. 5).

| | ACPA+ | | ACPA+ and ACPA− | |
|---|---|---|---|---|
| | RA sera | | RA sera | |
| Peptides | Sensitivity | Specificity | Sensitivity | Specificity |
| ACPA (CCP2) | N/A | N/A | 8/9 (89%) | 8/13 (62%) |
| cFbα 556-575 (1) | 8/8 (100%) | 2/5 (40%) | 8/9 (89%) | 10/13 (77%) |
| citFbβ 563-583 (2) | 6/8 (75%) | 4/5 (80%) | 6/9 (67%) | 12/13 (92%) |
| citH2A 1-20 (3) | 6/8 (75%) | 4/5 (80%) | 6/8 (75%) | 12/13 (92%) |
| (2) + (3) | 8/8 (100%) | 3/5 (60%) | 8/9 (89%) | 11/13 (85%) |

N/A: not applicable.

As shown in FIG. 5A, ACPA expression levels in serum and synovial fluid correlated to each other. As shown in FIG. 5B, ACPA expression levels in ACPA⁺ serum were enriched in the responder group. As shown in FIGS. 5C-5E, ACPA expression levels in ACPA⁺ serum were enriched in the responder group. As shown in FIGS. 5F-5H, ACPA expression levels in all serum samples, i.e., ACPA⁺ serum and ACAP⁻ serum, were enriched in the responder group.

Table 2 summarizes the sensitivity and specificity of the antibodies against specific citrullinated peptides in ACPA⁺ sera and in ACPA⁺ and ACPA⁻ sera to predict NI-0101 response. As shown in Table 2, the combination of citFbβ 563-583 and citH2A 1-20 is highly sensitive and highly specific in serum samples from RA patients.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying a patient suitable for therapy with an antagonist of Toll-like Receptor 4 (TLR4) and alleviating a symptom of a TLR4-related disorder, the method comprising detecting a level of expression for anti-citrullinated protein antibody (ACPA) and/or at least one antibody against specific citrullinated protein and/or peptide comprising an amino acid sequence selected from the group consisting of SEQ ID NO: 1, SEQ ID NO: 2, and SEQ ID NO: 3 in at least a first biological sample from a subject, comparing the detected level of ACPA and/or the at least one antibody against specific citrullinated protein and/or peptide to a control level of expression, and when the detected level is elevated, administering an anti-TLR4 antagonist in an amount sufficient to alleviate the symptom of the TLR4-related disorder to the subject.

2. The method of claim 1, wherein the method comprises detecting a level of expression for ACPA and/or a level of expression of an antibody against the peptide of SEQ ID NO: 1, an antibody against the peptide of SEQ ID NO: 2, an antibody against peptide of SEQ ID NO: 3, and any combinations thereof.

3. The method of claim 1, wherein the biological sample is or is derived from blood.

4. The method of claim 1, wherein the biological sample is serum.

5. The method of claim 1, wherein the biological sample is or is derived from synovial fluid.

6. The method of claim 1, wherein the method further comprises detecting a level of expression for ACPA and/or at least one antibody against a specific citrullinated peptide comprising an amino acid sequence selected from the group consisting of SEQ ID NO: 1, SEQ ID NO: 2, and SEQ ID NO: 3 in a second biological sample from the same subject.

7. The method of claim 6, wherein the first biological sample is or is derived from blood.

8. The method of claim 7, wherein the first biological sample is serum.

9. The method of claim 6, wherein the second biological sample is or is derived from synovial fluid.

10. The method of claim 1, wherein the anti-TLR4 antagonist is an anti-TLR4 antibody or immunologically active fragment thereof.

11. The method of claim 10, wherein the anti-TLR4 antibody or immunologically active fragment thereof comprises a variable heavy chain complementarity determining region 1 (VH CDR1) the amino acid sequence of GGYSWH (SEQ ID NO: 139); a VH CDR2 region comprising the amino acid sequence of YIHYSGYTDFNPSLKT (SEQ ID NO: 140); a VH CDR3 region comprising the amino acid sequence of KDPSDAFPY (SEQ ID NO: 141); a variable light chain complementarity determining region 1 (VL CDR1) region comprising the amino acid sequence of RASQSISDHLH (SEQ ID NO: 4); a VL CDR2 region comprising the amino acid sequence of YASHAIS (SEQ ID NO: 5); and a VL CDR3 region comprising the amino acid sequence of QQGHSFPLT (SEQ ID NO: 6).

12. The method of claim 10, wherein the anti-TLR4 antibody or immunologically active fragment thereof comprises the heavy chain variable amino acid sequence QVQLQESGPGLVKPSDTLSLTCAVSGYSITGGYSWHWIRQPPGKGLEWMGYIHYSGYT DFNPSLKTRITISRDTSKNQFSLKLSSVTAVDTAVYYCARKDPSDAFPYWGQGTLVTVSS (SEQ ID NO: 7) and the light chain variable amino acid sequence EIVLTQSPDFQSVTPKEKVTITCRASQSISDHLHWYQQKPDQSPKLLIKYASHAISGVPSR FSGSGSGTDFTLTINSLEAEDAATYYCQQGHSFPLTFGGGTKVEIK (SEQ ID NO: 8).

13. The method of claim 10, wherein the anti-TLR4 antibody or immunologically active fragment thereof comprises the heavy chain amino acid sequence MGWSWIFLFLLSGTAGVHCQVQLQESGPGLVKPSDTLSLTCAVSGYSITGGYSWHWIR QPPGKGLEWMGYIHYSGYTDFNPSLKTRITISRDTSKNQFSLKLSSVTAVDTAVYYCAR KDPSDAFPYWGQGTLVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNTKVDKRVE PKSCDKTHTCPPCPAPELLGGPSVFLFPPKPKDTLMISRTPEVTCVVVDVSHEDPEVKFN WYVDGVEVHNAKTKPREEQYNSTYRVVSVLTVLHQDWLNGKEYKCKVSSKAFPAPIE KTISKAKGQPREPQVYTLPPSREEMTKNQVSLTCLVKGFYPSDIAVEWESNGQPENNYK TTPPVLDSDGSFFLYSKLTVDKSRWQQGNVFSCSVMHEALHNHYTQKSLSLSPGK (SEQ ID NO: 9) and the light chain amino acid sequence MEWSWVFLFFLSVTTGVHSEIVLTQSPDFQSVTPKEKVTITCRASQSISDHLHWYQQKPD QSPKLLIKYASHAISGVPSRFSGSGSGTDFTLTINSLEAEDAATYYCQQGHSFPLTFGGGT KVEIKRTVAAPSVFIFPPSDEQLKSGTASVVCLLNNFYPREAKVQWKVDNALQSGNSQE SVTEQDSKDSTYSLSSTLTLSKADYEKHKVYACEVTHQGLSSPVTKSFNRGEC (SEQ ID NO: 10).

14. The method of claim 1, wherein the subject is human.

15. The method of claim 1, wherein the disorder is an autoimmune or inflammatory disorder.

16. The method of claim 1, wherein the disorder is associated with aberrant TLR4 signaling, elevated TLR4 ligand expression or activity, aberrant pro-inflammatory cytokine production, and combinations thereof.

17. The method of claim 1, wherein the disorder is rheumatoid arthritis (RA).

18. A method for diagnosing a TLR4-related disorder in a subject, the method comprising detecting a level of expression for anti-citrullinated protein antibody (ACPA) and/or at least one antibody against specific citrullinated protein and/or peptide comprising an amino acid sequence selected from the group consisting of SEQ ID NO: 1, SEQ ID NO: 2, and SEQ ID NO: 3 in at least a first biological sample from a subject, comparing the detected level of ACPA and/or the at least one antibody against specific citrullinated protein and/or peptide to a control level of expression, and when the detected level is elevated, diagnosing the subject with a TLR4-related disorder.

19. The method of claim 18, wherein the method comprises detecting a level of expression for ACPA and/or a level of expression of an antibody against the peptide of SEQ ID NO: 1, an antibody against the peptide of SEQ ID NO: 2, an antibody against peptide of SEQ ID NO: 3, and any combinations thereof.

20. The method of claim 18, wherein the biological sample is or is derived from blood.

21. The method of claim 18, wherein the biological sample is serum.

22. The method of claim 18, wherein the biological sample is or is derived from synovial fluid.

23. The method of claim 18, wherein the method further comprises detecting a level of expression for ACPA and/or at least one antibody against a specific citrullinated peptide comprising an amino acid sequence selected from the group consisting of SEQ ID NO: 1, SEQ ID NO: 2, and SEQ ID NO: 3 in a second biological sample from the same subject.

24. The method of claim 23, wherein the first biological sample is or is derived from blood.

25. The method of claim 24, wherein the first biological sample is serum.

26. The method of claim 23, wherein the second biological sample is or is derived from synovial fluid.

27. The method of claim 18, wherein the subject is human.

28. The method of claim 18, wherein the TLR4-related disorder is an autoimmune or inflammatory disorder.

29. The method of claim 18, wherein the TLR4-related disorder is associated with aberrant TLR4 signaling, elevated TLR4 ligand expression or activity, aberrant pro-inflammatory cytokine production, and combinations thereof.

30. The method of claim 18, wherein the TLR4-related disorder is rheumatoid arthritis (RA).


SEQUENCE LISTING (141 sequences)

SEQ ID NO: 1 - protein, 20 aa, Artificial Sequence
  chemically synthesized
NTKESSSHHPGIAEFPSXGK

SEQ ID NO: 2 - protein, 21 aa, Artificial Sequence
  chemically synthesized
HHPGIAEFPSXGKSSSYSKQF

SEQ ID NO: 3 - protein, 20 aa, Artificial Sequence
  chemically synthesized
MSGXGKQGGKAXAKAKSXSS

SEQ ID NO: 4 - protein, 11 aa, Artificial Sequence
  chemically synthesized
RASQSISDHLH

SEQ ID NO: 5 - protein, 7 aa, Artificial Sequence
  chemically synthesized
YASHAIS

SEQ ID NO: 6 - protein, 9 aa, Artificial Sequence
  chemically synthesized
QQGHSFPLT

SEQ ID NO: 7 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSDAFPYWGQGTLVTVSS

SEQ ID NO: 8 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIK

SEQ ID NO: 9 - protein, 467 aa, Artificial Sequence
  chemically synthesized
MGWSWIFLFLLSGTAGVHCQVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSDAFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  chemically synthesized
MEWSWVFLFFLSVTTGVHSEIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 1404 bp, Artificial Sequence
  chemically synthesized
atgggatggagctggatctttctcttcctcctgtcaggaactgcaggtgtacattgccaggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaaatgcaaggtctccagtaaagctttccctgcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 12 - DNA, 702 bp, Artificial Sequence
  chemically synthesized
atggaatggagctgggtctttctcttcttcctgtcagtaactacaggtgtccactccgaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 4 aa, Artificial Sequence
  chemically synthesized
SKAF

SEQ ID NO: 14 - protein, 9 aa, Artificial Sequence
  chemically synthesized
GXPIXXGYS

SEQ ID NO: 15 - protein, 9 aa, Artificial Sequence
  chemically synthesized
GYSITGGYS

SEQ ID NO: 16 - protein, 9 aa, Artificial Sequence
  chemically synthesized
GFPIRYGYS

SEQ ID NO: 17 - protein, 9 aa, Artificial Sequence
  chemically synthesized
GYPIRFGYS

SEQ ID NO: 18 - protein, 9 aa, Artificial Sequence
  chemically synthesized
GYPIRHGYS

SEQ ID NO: 19 - protein, 9 aa, Artificial Sequence
  chemically synthesized
GFPIGQGYS

SEQ ID NO: 20 - protein, 9 aa, Artificial Sequence
  chemically synthesized
GYPIWGGYS

SEQ ID NO: 21 - protein, 9 aa, Artificial Sequence
  chemically synthesized
GYPIGGGYS

SEQ ID NO: 22 - protein, 7 aa, Artificial Sequence
  chemically synthesized
IHYSGYT

SEQ ID NO: 23 - protein, 11 aa, Artificial Sequence
  chemically synthesized
ARKDSGXXXPY

SEQ ID NO: 24 - protein, 11 aa, Artificial Sequence
  chemically synthesized
ARKDSGNYFPY

SEQ ID NO: 25 - protein, 11 aa, Artificial Sequence
  chemically synthesized
ARKDSGRLLPY

SEQ ID NO: 26 - protein, 11 aa, Artificial Sequence
  chemically synthesized
ARKDSGKWLPY

SEQ ID NO: 27 - protein, 11 aa, Artificial Sequence
  chemically synthesized
ARKDSGHLMPY

SEQ ID NO: 28 - protein, 11 aa, Artificial Sequence
  chemically synthesized
ARKDSGHNYPY

SEQ ID NO: 29 - protein, 11 aa, Artificial Sequence
  chemically synthesized
ARKDSGKNFPY

SEQ ID NO: 30 - protein, 11 aa, Artificial Sequence
  chemically synthesized
ARKDSGQLFPY

SEQ ID NO: 31 - protein, 11 aa, Artificial Sequence
  chemically synthesized
ARKDSGHNLPY

SEQ ID NO: 32 - protein, 11 aa, Artificial Sequence
  chemically synthesized
ARKDSGDYFPY

SEQ ID NO: 33 - protein, 11 aa, Artificial Sequence
  chemically synthesized
ARKDSGRYWPY

SEQ ID NO: 34 - protein, 6 aa, Artificial Sequence
  chemically synthesized
QSISDH

SEQ ID NO: 35 - protein, 3 aa, Artificial Sequence
  chemically synthesized
YAS

SEQ ID NO: 36 - protein, 9 aa, Artificial Sequence
  chemically synthesized
QQGXXXPXT

SEQ ID NO: 37 - protein, 9 aa, Artificial Sequence
  chemically synthesized
QQGNDFPVT

SEQ ID NO: 38 - protein, 9 aa, Artificial Sequence
  chemically synthesized
QQGYDEPFT

SEQ ID NO: 39 - protein, 9 aa, Artificial Sequence
  chemically synthesized
QQGYDFPFT

SEQ ID NO: 40 - protein, 9 aa, Artificial Sequence
  chemically synthesized
QQGYDYPFT

SEQ ID NO: 41 - protein, 9 aa, Artificial Sequence
  chemically synthesized
QQGYEFPFT

SEQ ID NO: 42 - protein, 118 aa, Artificial Sequence
  chemically synthesized
QVQLQESGPGLVKPSDTLSLTCAVSGYSIXGGYSWHWIRQPPGKGLEWXGYIHYSGYTDFNPSLKTRXTXSRDTSKNQFSLKLSSVTAVDTAVYYCARKDPSDGFPYWGQGTLVTVSS

SEQ ID NO: 43 - protein, 118 aa, Artificial Sequence
  chemically synthesized
EVQLVESGGGLVQPGGSLRLSCAXSGYSITGGYSWHWVRQAPGKGLEWXSYIHYSGYTDFNPSLKTRFTISRDNSKNTXYLQMNSLRAEDTAVYYCARKDPSDGFPYWGQGTLVTVSS

SEQ ID NO: 44 - protein, 107 aa, Artificial Sequence
  chemically synthesized
EIVLTQSPATLSLSPGERATLSCRASQSISDHLHWYQQKPGQAPRLLIXYASHAISGIPARFSGSGSGTDFTLTISSLEPEDFAVYYCQNGHSFPLTFGGGTKVEIK

SEQ ID NO: 45 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQNGHSFPLTFGGGTKVEIK

SEQ ID NO: 46 - protein, 120 aa, Artificial Sequence
  chemically synthesized
QVQLVQSGAEVKKPGSSVKVSCKASGFNIKDSYIHWVRQAPGQGLEWXGWTDPENVNSIYDPRFQGRVTITADXSTSTAYXELSSLRSEDTAVYYCARGYNGVYYAMDYWGQGTTVTVSS

SEQ ID NO: 47 - protein, 5 aa, Artificial Sequence
  chemically synthesized
DSYIH

SEQ ID NO: 48 - protein, 17 aa, Artificial Sequence
  chemically synthesized
WTDPENVNSIYDPRFQG

SEQ ID NO: 49 - protein, 11 aa, Artificial Sequence
  chemically synthesized
GYNGVYYAMDY

SEQ ID NO: 50 - protein, 106 aa, Artificial Sequence
  chemically synthesized
EIVLTQSPATLSLSPGERATLSCSASSSVIYMHWYQQKPGQAPRLLIYRTYNLASGIPARFSGSGSGTDXTLTISSLEPEDFAVYYCHQWSSFPYTFGQGTKVEIK

SEQ ID NO: 51 - protein, 10 aa, Artificial Sequence
  chemically synthesized
SASSSVIYMH

SEQ ID NO: 52 - protein, 7 aa, Artificial Sequence
  chemically synthesized
RTYNLAS

SEQ ID NO: 53 - protein, 9 aa, Artificial Sequence
  chemically synthesized
HQWSSFPYT

SEQ ID NO: 54 - protein, 121 aa, Artificial Sequence
  chemically synthesized
QVTLRESGPALVKPTQTLTLTCTFSGFSLXTYNIGVGWIRQPPGKALEWLAHIWWNDNIYYNTVLKSRLTXSKDTSKNQVVLTMTNMDPVDTATYYCXRMAEGRYDAMDYWGQGTLVTVSS

SEQ ID NO: 55 - protein, 7 aa, Artificial Sequence
  chemically synthesized
TYNIGVG

SEQ ID NO: 56 - protein, 16 aa, Artificial Sequence
  chemically synthesized
HIWWNDNIYYNTVLKS

SEQ ID NO: 57 - protein, 11 aa, Artificial Sequence
  chemically synthesized
MAEGRYDAMDY

SEQ ID NO: 58 - protein, 121 aa, Artificial Sequence
  chemically synthesized
EVQLVESGGGLVQPGGSLRLSCAXSGFSLTTYNIGVGWVRQAPGKGLEWXSHIWWNDNIYYNTVLKSRLTXSXDNSKNTXYLQMNSLRAEDTAVYYCXRMAEGRYDAMDYWGQGTLVTVSS

SEQ ID NO: 59 - protein, 7 aa, Artificial Sequence
  chemically synthesized
TYNIGVG

SEQ ID NO: 60 - protein, 16 aa, Artificial Sequence
  chemically synthesized
HIWWNDNIYYNTVLKS

SEQ ID NO: 61 - protein, 11 aa, Artificial Sequence
  chemically synthesized
MAEGRYDAMDY

SEQ ID NO: 62 - protein, 107 aa, Artificial Sequence
  chemically synthesized
DIQMTQSPSSVSASVGDRVTITCRASQDITNYLNWYQQKPGKAPKLLIYYTSKLHSGVPSRFSGSGSGTDXTLTISSLQPEDFATYXCQQGNTFPWTFGGGTKVEIK

SEQ ID NO: 63 - protein, 11 aa, Artificial Sequence
  chemically synthesized
RASQDITNYLN

SEQ ID NO: 64 - protein, 7 aa, Artificial Sequence
  chemically synthesized
YTSKLHS

SEQ ID NO: 65 - protein, 9 aa, Artificial Sequence
  chemically synthesized
QQGNTFPWT

SEQ ID NO: 66 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttcc

SEQ ID NO: 67 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDPSEGFPYWGQGTLVTVSS

SEQ ID NO: 68 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgagggatttccttactggggccaagggactctggtcactgtctcttcc

SEQ ID NO: 69 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQNSHSFPLTFGGGTKVEIK

SEQ ID NO: 70 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagaatagtcacagttttccgctcactttcggcggagggaccaaggtggagatcaaa

SEQ ID NO: 71 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaa

SEQ ID NO: 72 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQNSSSFPLTFGGGTKVEIK

SEQ ID NO: 73 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagaatagtagtagttttccgctcactttcggcggagggaccaaggtggagatcaaa

SEQ ID NO: 74 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQSHSFPLTFGGGTKVEIK

SEQ ID NO: 75 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagagtcacagttttccgctcactttcggcggagggaccaaggtggagatcaaa

SEQ ID NO: 76 - protein, 839 aa, Artificial Sequence
  chemically synthesized
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 77 - protein, 826 aa, Artificial Sequence
  chemically synthesized
MTSALRLAGTLIPAMAFLSCVRPESWEPCVEVVPNITYQCMELKFYKIPDNIPFSTKNLDLSFNPLRHLGSYSFLRFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQNIYCKDLQVLHQMPLSNLSLDLSLNPINFIQPGAFKEIRLHKLTLRSNFDDLNVMKTCIQGLAGLEVHRLVLGEFRNERNLEEFDKSSLEGLCNLTIEEFRLTYLDCYLDNIIDLFNCLANVSSFSLVSVNIKRVEDFSYNFRWQHLELVNCKFEQFPTLELKSLKRLTFTANKGGNAFSEVDLPSLEFLDLSRNGLSFKGCCSQSDFGTTSLKYLDLSFNDVITMSSNFLGLEQLEHLDFQHSNLKQMSQFSVFLSLRNLIYLDISHTHTRVAFNGIFDGLLSLKVLKMAGNSFQENFLPDIFTDLKNLTFLDLSQCQLEQLSPTAFDTLNKLQVLNMSHNNFFSLDTFPYKCLPSLQVLDYSLNHIMTSNNQELQHFPSSLAFLNLTQNDFACTCEHQSFLQWIKDQRQLLVEAERMECATPSDKQGMPVLSLNITCQMNKTIIGVSVFSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGQHIFWRRLRKALLDGKSWNPEEQ

SEQ ID NO: 78 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 79 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 80 - DNA, 321 bp, Artificial Sequence; identical to SEQ ID NO: 71
  chemically synthesized

SEQ ID NO: 81 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccggccgcctcctcccttactggggccaagggactctggtcactgtctcttcc

SEQ ID NO: 82 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGRLLPYWGQGTLVTVSS

SEQ ID NO: 83 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggcaagtggttgccttactggggccaagggactctggtcactgtctcttcc

SEQ ID NO: 84 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGKWLPYWGQGTLVTVSS

SEQ ID NO: 85 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcgggcacctcatgccttactggggccaagggactctggtcactgtctcttcc

SEQ ID NO: 86 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHLMPYWGQGTLVTVSS

SEQ ID NO: 87 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccgggcacaactacccttactggggccaagggactctggtcactgtctcttcc

SEQ ID NO: 88 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHNYPYWGQGTLVTVSS

SEQ ID NO: 89 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggcaagaacttcccttactggggccaagggactctggtcactgtctcttcc

SEQ ID NO: 90 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGKNFPYWGQGTLVTVSS

SEQ ID NO: 91 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggccagttgttcccttactggggccaagggactctggtcactgtctcttcc

SEQ ID NO: 92 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGQLFPYWGQGTLVTVSS

SEQ ID NO: 93 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatagcggccacaacttgccttactggggccaagggactctggtcactgtctcttcc

SEQ ID NO: 94 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGHNLPYWGQGTLVTVSS

SEQ ID NO: 95 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcgactacttcccttactggggccaagggactctggtcactgtctcttcc

SEQ ID NO: 96 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGDYFPYWGQGTLVTVSS

SEQ ID NO: 97 - DNA, 354 bp, Artificial Sequence
  chemically synthesized
caggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattccgggcggtactggccttactggggccaagggactctggtcactgtctcttcc

SEQ ID NO: 98 - protein, 118 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGRYWPYWGQGTLVTVSS

SEQ ID NO: 99 - DNA, 354 bp, Artificial Sequence
  chemically synthesized
caggtgcagcttcaggagtccggcccaggactggtgaagccttcggacaccctgtccctcacctgcgctgtctctggtttcccgatccgctacgggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 100 - protein, 118 aa, Artificial Sequence
  chemically synthesized
QVQLQESGPGLVKPSDTLSLTCAVSGFPIRYGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 101 - DNA, 354 bp, Artificial Sequence
  chemically synthesized
caggtgcagcttcaggagtccggcccaggactggtgaagccttcggacaccctgtccctcacctgcgctgtctctggttacccgatccggttcggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 102 - protein, 118 aa, Artificial Sequence
  chemically synthesized
QVQLQESGPGLVKPSDTLSLTCAVSGYPIRFGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 103 - DNA, 354 bp, Artificial Sequence
  chemically synthesized
caggtgcagcttcaggagtccggcccaggactggtgaagccttcggacaccctgtccctcacctgcgctgtctctggttaccccatccggcacgggtacagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 104 - protein, 118 aa, Artificial Sequence
  chemically synthesized
QVQLQESGPGLVKPSDTLSLTCAVSGYPIRHGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 105 - DNA, 354 bp, Artificial Sequence
  chemically synthesized
caggtgcagcttcaggagtccggcccaggactggtgaagccttcggacaccctgtccctcacctgcgctgtctctggtttcccgatcggccaggggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 106 - protein, 118 aa, Artificial Sequence
  chemically synthesized
QVQLQESGPGLVKPSDTLSLTCAVSGFPIGQGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 107 - DNA, 363 bp, Artificial Sequence
  chemically synthesized
caggtgcagcttcaggagtccggcccaggactggtgaagccttcggacaccctgtccctcacctgcgctgtctctggttacccgatctgggggggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttccgcctccacc

SEQ ID NO: 108 - protein, 118 aa, Artificial Sequence
  chemically synthesized
QVQLQESGPGLVKPSDTLSLTCAVSGYPIWGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 109 - DNA, 354 bp, Artificial Sequence
  chemically synthesized
caggtgcagcttcaggagtccggcccaggactggtgaagccttcggacaccctgtccctcacctgcgctgtctctggttaccccatcggcggcggctatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttcc

SEQ ID NO: 110 - protein, 118 aa, Artificial Sequence
  chemically synthesized
QVQLQESGPGLVKPSDTLSLTCAVSGYPIGGGYSWHWIRQPPGKGLEWMGYIHYSGYTDFNPSLKTRITISRDTSKNQFSLKLSSVTAVDTAVYYCARKDSGNYFPYWGQGTLVTVSS

SEQ ID NO: 111 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcaggggaacgacttcccggtgactttcggcggagggaccaaggtggagatcaaa

SEQ ID NO: 112 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGNDFPVTFGGGTKVEIK

SEQ ID NO: 113 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcaggggtacgacgagccgttcactttcggcggagggaccaaggtggagatcaaa

SEQ ID NO: 114 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGYDEPFTFGGGTKVEIK

SEQ ID NO: 115 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgacttcccgttgactttcggcggagggaccaaggtggagatcaaa

SEQ ID NO: 116 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGYDFPLTFGGGTKVEIK

SEQ ID NO: 117 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgactacccgctcactttcggcggagggaccaaggtggagatcaaa

SEQ ID NO: 118 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGYDYPLTFGGGTKVEIK

SEQ ID NO: 119 - DNA, 321 bp, Artificial Sequence
  chemically synthesized
gaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggctacgagttcccgttgactttcggcggagggaccaaggtggagatcaaa

SEQ ID NO: 120 - protein, 107 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGYEFPLTFGGGTKVEIK

SEQ ID NO: 121 - DNA, 321 bp, Artificial Sequence; identical to SEQ ID NO: 111
  chemically synthesized

SEQ ID NO: 122 - protein, 107 aa, Artificial Sequence; identical to SEQ ID NO: 112
  chemically synthesized

SEQ ID NO: 123 - DNA, 321 bp, Artificial Sequence; identical to SEQ ID NO: 115
  chemically synthesized

SEQ ID NO: 124 - protein, 107 aa, Artificial Sequence; identical to SEQ ID NO: 116
  chemically synthesized

SEQ ID NO: 125 - DNA, 321 bp, Artificial Sequence; identical to SEQ ID NO: 117
  chemically synthesized

SEQ ID NO: 126 - protein, 107 aa, Artificial Sequence; identical to SEQ ID NO: 118
  chemically synthesized

SEQ ID NO: 127 - DNA, 321 bp, Artificial Sequence; identical to SEQ ID NO: 119
  chemically synthesized

SEQ ID NO: 128 - protein, 107 aa, Artificial Sequence; identical to SEQ ID NO: 120
  chemically synthesized

SEQ ID NO: 129 - DNA, 1398 bp, Artificial Sequence
  chemically synthesized
atggaatggagctgggtctttctcttcttcctgtcagtaactacaggtgtccaccaggtgcagcttcaggagtccggcccaggactggtgaagccttcggacaccctgtccctcacctgcgctgtctctggttactccatcaccggtggttatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagatccgtccgacgcctttccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacagtctcgtggaactcaggagccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatctcgggaggagatgaccaagaaccaggtcagcctgacttgcctggtcaaaggcttctatcccagcgacatcgccgtggagtgggagagcaacgggcagccggagaacaactacaagaccacgcctcccgtgctggactccgacggctccttcttcctctatagcaagctcaccgtggacaagtccaggtggcagcaggggaacgtcttctcatgctccgtgatgcatgaggctctgcacaaccactacacgcagaagagcctctccctgtctccgggttaa

SEQ ID NO: 130 - protein, 447 aa, Artificial Sequence
  chemically synthesized
QVQLQESGPGLVKPSDTLSLTCAVSGYSITGGYSWHWIRQPPGKGLEWMGYIHYSGYTDFNPSLKTRITISRDTSKNQFSLKLSSVTAVDTAVYYCARKDSGNYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - DNA, 705 bp, Artificial Sequence
  chemically synthesized
atgagtgtgcccactcaggtcctggggttgctgctgctgtggcttacagatgccagatgtgaaattgtgttgacgcagtctccagactttcagtctgtgactccaaaggaaaaagtcaccatcacctgcagggccagtcagagtatcagcgaccacttacactggtaccaacagaaacctgatcagtctcccaagctcctcatcaaatatgcttcccatgccatttctggggtcccatcgaggttcagtggcagtgggtctgggacagacttcactctcaccatcaatagcctagaggctgaagatgctgcaacgtattactgtcagcagggtcacagttttccgctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 132 - protein, 214 aa, Artificial Sequence
  chemically synthesized
EIVLTQSPDFQSVTPKEKVTITCRASQSISDHLHWYQQKPDQSPKLLIKYASHAISGVPSRFSGSGSGTDFTLTINSLEAEDAATYYCQQGHSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 1398 bp, Artificial Sequence
  chemically synthesized
atggaatggagctgggtctttctcttcttcctgtcagtaactacaggtgtccaccaggtgcagcttcaggagtccggcccaggactggtgaagccttcggacaccctgtccctcacctgcgctgtctctggtttcccgatccgctacgggtatagctggcactggatacggcagcccccagggaagggactggagtggatggggtatatccactacagtggttacactgacttcaacccctccctcaagactcgaatcaccatatcacgtgacacgtccaagaaccagttctccctgaagctgagctctgtgaccgctgtggacactgcagtgtattactgtgcgagaaaagattcgggcaactacttcccttactggggccaagggactctggtcactgtctcttccgcctccaccaagggcccatcggtcttccccctggcaccctcctccaagagcacctctgggggcacagcggccctgggctgcctggtcaaggactacttccccgaaccggtgacagtctcgtggaactcaggagccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtataccctgcccccatctcgggaggagatgaccaagaaccaggtcagcctgacttgcctggtcaaaggcttctatcccagcgacatcgccgtggagtgggagagcaacgggcagccggagaacaactacaagaccacgcctcccgtgctggactccgacggctccttcttcctctatagcaagctcaccgtggacaagtccaggtggcagcaggggaacgtcttctcatgctccgtgatgcatgaggctctgcacaaccactacacgcagaagagcctctccctgtctccgggttaa

SEQ ID NO: 134 - protein, 446 aa, Artificial Sequence
  chemically synthesized
QVQLQESGPGLVKPSDTLSLTCAVSGFPIRYGYSWHWIRQPPGKGLEWMGYIHYSGYTDFNPLKTRITISRDTSKNQFSLKLSSVTAVDTAVYYCARKDSGNYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - DNA, 705 bp, Artificial Sequence; identical to SEQ ID NO: 131
  chemically synthesized

SEQ ID NO: 136 - protein, 214 aa, Artificial Sequence; identical to SEQ ID NO: 132
  chemically synthesized

SEQ ID NO: 137 - protein, 9 aa, Artificial Sequence
  chemically synthesized
KDPSDGFPY

SEQ ID NO: 138 - protein, 9 aa, Artificial Sequence
  chemically synthesized
QNGHSFPLT

SEQ ID NO: 139 - protein, 6 aa, Artificial Sequence
  chemically synthesized
GGYSWH

SEQ ID NO: 140 - protein, 16 aa, Artificial Sequence
  chemically synthesized
YIHYSGYTDFNPSLKT

SEQ ID NO: 141 - protein, 9 aa, Artificial Sequence
  chemically synthesized
KDPSDAFPY